(19) United States
(12) Patent Application Publication
(10) Pub. No.: US 2024/0075141 A1
(43) Pub. Date: Mar. 7, 2024

(54) CHIMERIC RECEPTOR CONSTRUCTS FOR NK CELLS

(71) Applicant: Wugen, Inc., Saint Louis, MO (US)

(72) Inventors: You ZHOU, Creve Coeur, MO (US); Matthew COOPER, Saint Louis, MO (US); Elizabeth SCHRAMM, O'Fallon, IL (US)

(21) Appl. No.: 18/186,133

(22) Filed: Mar. 17, 2023

Related U.S. Application Data
(60) Provisional application No. 63/321,359, filed on Mar. 18, 2022.

(51) Int. Cl. A61K 39/00; A61P 35/00; C07K 14/705; C07K 14/715; C07K 14/725; C07K 14/735; C07K 16/10; C07K 16/28

(52) U.S. Cl. CPC A61K 39/4631; A61K 39/4613; A61P 35/00; C07K 14/705; C07K 14/70503; C07K 14/70507; C07K 14/7051; C07K 14/70517; C07K 14/70521; C07K 14/70535; C07K 14/70596; C07K 14/7155; C07K 16/1063; C07K 16/2803; C07K 16/2806; C07K 16/2809; C07K 16/2818; C07K 16/2851; C07K 16/2863; C07K 16/2866; C07K 16/2878; C07K 16/2896; C07K 2317/622; C07K 2319/02; C07K 2319/03; C07K 2319/30

(57) ABSTRACT

Among the various aspects of the present disclosure is the provision of improved chimeric receptor constructs for NK cells, NK cells containing such receptors and methods of making and using same.

### CROSS-REFERENCE TO RELATED APPLICATIONS

This application claims the benefit of U.S. Provisional Application No. 63/321,359, filed Mar. 18, 2022, the contents of all of which is herein incorporated by reference in their entirety.

### STATEMENT REGARDING SEQUENCE LISTING

The Sequence Listing XML associated with this application is provided in XML file format and is hereby incorporated by reference into the specification. The name of the XML file containing the Sequence Listing XML is WUGE_002_01US_ST26.xml. The XML file is 66,645 bytes, and created on Mar. 17, 2023, and is being submitted electronically via USPTO Patent Center.

### FIELD OF THE INVENTION

The present disclosure relates generally to modified natural killer (NK) cells containing chimeric receptors and methods of making and using the same.

### BACKGROUND OF THE INVENTION

A Chimeric Antigen Receptor (CAR) is generally made up of an extracellular domain that facilitates recognition of a target antigen of interest (e.g., a disease-associated antigen), a transmembrane domain and one or more intracellular signaling domains. CARs have been widely reported in field of T cell immunotherapy and various domains that can be used in CAR T cells have been described. However, much less is known about optimizing CAR functionality and effectiveness in NK cells. To date, most CAR designs in NK cells have made use of intracellular signaling domains that have been used in the development of CAR T cells. However, it is becoming increasingly clear that such domains are not optimal for use in NK cells, in part due to the unique signaling machinery present in NK cells. As a result, there is an important unmet need for identifying CAR domains that can be more effectively used in NK cells for therapeutic and other applications. As described herein, the present disclosure meets this need and offers other related advantages.

### SUMMARY OF THE INVENTION

According to one aspect of the present disclosure, there are provided chimeric antigen receptors (CARs) capable of being expressed and functioning in NK cells, where the CARs comprise at least two of a CD79A intracellular signaling domain, a CD79B intracellular signaling domain, a 2B4 intracellular signaling domain and a DAP10 intracellular signaling domain.

According to another aspect of the present disclosure, there are provided CARs capable of being expressed and functioning in NK cells, where the CARs comprises each of a CD79A intracellular signaling domain, a CD79B intracellular signaling domain and a 2B4 intracellular signaling domain.

According to another aspect of the present disclosure, there are provided CARs capable of being expressed and functioning in NK cells, where the CARs comprises each of a CD79A intracellular signaling domain, a CD79B intracellular signaling domain and a DAP10 intracellular signaling domain.

In some embodiments of the CARs of the disclosure, the CD79A intracellular signaling domain is encoded by a sequence set forth in SEQ ID NO: 1 (WT), a sequence set forth in SEQ ID NO: 2 (CD79A (S197A, 5203A, T209V), or a is a functional fragment or variant thereof having at least 80%, 85%, 90%, 95% or 99% identity to a sequence encoded by SEQ ID NO: 1 or 2.

In some embodiments of the CARs of the disclosure, the CD79B intracellular signaling domain comprises a sequence encoded by SEQ ID NO:3, or is a functional fragment or variant thereof having at least 80%, 85%, 90%, 95% or 99% identity to a sequence encoded by SEQ ID NO: 3.

In some embodiments of the CARs of the disclosure, the 2B4 intracellular signaling domain comprises a sequence encoded by SEQ ID NO: 4, or is a functional fragment or variant thereof having at least 80%, 85%, 90%, 95% or 99% identity to a sequence encoded by SEQ ID NO: 4.

In some embodiments of the CARs of the disclosure, the DAP10 intracellular signaling domain comprises a sequence encoded by SEQ ID NO: 5, or is a functional fragment or variant thereof having at least 80%, 85%, 90%, 95% or 99% identity to a sequence encoded by SEQ ID NO: 5.

In some embodiments of the CARs of the disclosure, the CAR comprises an extracellular domain capable of binding a target polypeptide. In particular embodiments, the extracellular domain comprises an scFv sequence capable of binding a target polypeptide. In other particular embodiments, the scFv sequence binds a target polypeptide selected from the group consisting of CD2, CD5, CD7, MSLN, CEA, PSMA, CD19, CD28, CD3, CD33, CD38, CD138, CLL-1, C-KIT, CD123, CD133, CD20, BCMA, EGFR, CD3, CD4, BAFF-R, EGFR, HER2, GD2 gp120 and gp41.

In other embodiments of the CARs of the disclosure, the extracellular domain that is present in the CAR comprises an Fc receptor sequence. In particular embodiments, the Fc receptor sequence comprises a sequence chosen or derived from a CD16 receptor sequence, a CD32 receptor sequence or a CD64 receptor sequence. In other particular embodiments, the Fc receptor sequence is chosen or derived from a CD16 receptor sequence having a S197P mutation.

In other particular embodiments of the CARs of the disclosure, the extracellular domain that is present in the CAR comprises an extracellular domain of CD3e or CD28 (e.g., to enable the use of bispecific antibodies used to engage T cells).

In other embodiments of the CARs of the disclosure, the CAR comprises a transmembrane domain. In more particular embodiments, the transmembrane domain is a transmembrane domain sequence chosen or derived from CD28, CD16, CD32, CD64, NKG2D, FcγRIIIa, NKp44, NKp30, NKp46, actKIR, NKG2C, CD8α, and IL-15. In a particular embodiment, the transmembrane domain is chosen or derived from a CD16 transmembrane domain sequence.

In some embodiments of the CARs of the disclosure, the CAR construct, in addition to containing the CD79A, CD79B and/or 2B4 intracellular signaling domains, may further comprise one or more additional intracellular signaling domains, such as one chosen or derived from an intracellular signaling domain sequence of CD132, CD137/41BB, DNAM-1, NKp80, 2B4, NTBA, CRACC, CD2, CD27, integrins, IL-15R, IL-18R, IL-12R, IL-21R and IRE1a.

In some embodiments of the CARs of the disclosure, the CAR construct also comprises a hinge domain sequence, such as a hinge domain sequence chosen or derived from a CD8a hinge domain sequence, an NKG2 hinge domain sequence or a TMα hinge domain sequence.

In some embodiments of the CARs of the disclosure, the CAR is expressed under the control of a promoter that is transcriptionally active in NK cells. In more particular embodiments, the promoter is an MND promoter.

In some embodiments of the CARs of the disclosure, the CAR further comprises a P2A truncated CD34 protein on the terminal end of the chimeric receptor.

According to another aspect of the present disclosure, there are provided vectors containing nucleic acid molecules encoding the CARs as described herein. In some embodiments, the vector is a viral vector. In particular embodiments, the viral vector is a lentiviral vector or a retroviral vector. In other particular embodiments, the vector is a non-viral vector (e.g., a transposon).

According to another aspect of the present disclosure, there are provided genetically modified NK cells comprising (i) a CAR as described herein or (ii) a vector encoding or containing a CAR sequence as described herein.

In some embodiments, the genetically modified NK cell of the present disclosure is one that has been modified to be deficient for NKG2A and/or CD8 expression, activity or signaling.

In some embodiments, the genetically modified NK cell is an NK cell is that is derived from cord blood, peripheral blood, an immortalized cell line or an iPSC.

In particular embodiments, the genetically modified NK cell according to the present disclosure is a memory-like NK cell, such as a cytokine-induced memory-like NK cell.

According to another aspect of the present disclosure, there are provided methods of inducing an immune response to a disease in a subject in need thereof comprising administering to the subject (i) a CAR as described herein, (ii) a vector as described herein or (iii) a genetically modified cell as described herein. In some embodiments, the disease is cancer, an autoimmune condition or an infectious disease.

### DESCRIPTION OF THE DRAWINGS

FIG. 1 shows illustrative CAR molecules prepared according to the present disclosure.

FIG. 2 shows that a CAR containing intracellular signaling domains derived from CD79A, CD79B and 2B4 gave rise to enhanced killing of the pancreatic tumor cell line Aspc1. NK cells were purified from human PBMCs via CD56 enrichment. NK cells were cultured with NKMacs media (Miltenyi) in the presence of WuExpand (a feeder cell-free expansion system as described in WO2020/047473) and transduced with lentivirus encoding the designated constructs. After 14 days of culture, transduced NK cells were seeded into a culture vessel containing AsPC1 tumor cells and placed in an Incucyte plate reader at various effector to target ratios to evaluate specific lysis capacity. In certain conditions, additional targets were added at 72 hours after initial challenge for a “rechallenge” assay to determine long-term killing capacity.

FIG. 3 shows that NK cells transduced with CARs containing intracellular signaling domains derived from CD79A, CD79B and 2B4 demonstrated an unexpected ability to self-enrich during manufacturing. NK cells were purified from human PBMCs via CD56 enrichment. NK cells were cultured with NKMacs media in the presence of WuExpand and transduced with lentivirus encoding the designated constructs and a truncated CD34 tag. Two 2 days after lentiviral transduction (D4), cells were sampled and flow cytometry using CD34 and CD56 fluorophore conjugated antibodies was performed. NK cells were maintained in culture for 12 additional days (D14) and sampled again using Flow cytometry.

FIG. 4 shows that NK cells transduced with a CAR that used standard intracellular signaling domains used in T cell CARs were highly contaminated with T cells at the end of manufacture, despite having a low T cell percentage initially. NK cells were purified from human PBMCs via CD56 enrichment. NK cells were cultured with NKMacs media in the presence of WuExpand and transduced with lentivirus encoding the designated CAR constructs. Cells were assayed by Flow Cytometry for the presence of CD3+ CD56− T cells at the start of manufacturing (DO), at the end of manufacturing (D14) and after in vitro tumor challenge.

FIG. 5 shows that that enhanced ADCC-enabled NK cells (E-ADCC-enabled NK cells) containing a CAR having an extracellular domain comprising a CD16 receptor and intracellular signaling domains derived from CD79A, CD79B and 2B4 exhibited increased cytotoxicity towards SKOV3 cancer cells. NK cells were purified from human PBMCs via CD56 enrichment. NK cells were cultured with NKMacs media in the presence of WuExpand and transduced with lentivirus encoding the designated eADCC-CAR constructs. After 14 days of culture, transduced NK cells were seeded into a culture vessel containing SKOV3 tumor cells and placed in an Incucyte plate reader in the presence of either Trastuzumab (3 ug/mL) or Isotype control IgG1 (3 ug/mL) to evaluate specific lysis capacity.

FIG. 6 shows that that ADCC-enabled NK cells containing a CAR having an extracellular domain comprising a CD16 receptor and intracellular signaling domains derived from CD79A, CD79B and 2B4 exhibited increased cytotoxicity towards SCC26 cancer cells. NK cells were purified from human PBMCs via CD56 enrichment. NK cells were cultured with NKMacs media in the presence of WuExpand and transduced with lentivirus encoding the designated eADCC-CAR constructs. After 14 days of culture, transduced NK cells were seeded into a culture vessel containing SCC25 tumor cells and placed in an Incucyte plate reader in the presence of either Cetuximab (1 ug/mL) or Isotype control IgG1 (1 ug/mL) to evaluate specific lysis capacity.

FIG. 7 provides a schematic diagram showing a chimeric receptor-expressing NK cell using bispecific T cell engagers (BiTEs) to target cancer cells.

FIG. 8 provides a graph showing the number of available, clinical bispecific T cell engagers (TCEs) compared to the number of NK cell engagers (NKEs).

FIG. 9A provides a chart showing the number of exemplary clinical NKEs that can be used in combination with CD3-NK cells, their tumor target antigens, and the category of the tumor targets.

FIG. 9B provides a chart showing the number of exemplary clinical TCEs that can be used in combination with CD3-NK cells, their tumor target antigens, and the category of the tumor targets.

FIG. 10 provides a set of schematic diagrams showing exemplary embodiments of killing target cells using TCEs with endogenous T cells, using TCEs with endogenous T cells and CD3-NK cells, and using TCEs in combination with CD3-NK cells after conditioning to eliminate endogenous T cells.

FIG. 11 provides a schematic diagram for stabilizing cell surface expression of CD3e as a single chain variable fragment (Fv) by co-expression with other CD3 members (1) through a linker (2) through altering the highly conserved CXXC motif found on the membrane proximal end of CD3e, CD3g or CD3d (3) and through varying the position of CD3e as the membrane proximal or distal domain within the extracellular region (4).

FIG. 12A and FIG. 12B provide schematic diagrams showing chimeric receptor constructs and bicistronic chimeric receptor constructs for expression of CD3e on the cell surface.

FIG. 13A provides a diagram showing two chimeric receptor constructs WU76E and WU71A. WU76E is a bicistronic receptor expressing a first chimeric receptor with a CD3e extracellular domain, a CD3e transmembrane domain, and a CD3zeta intracellular domain; and a second chimeric receptor with a CD3g extracellular domain, a CD3g transmembrane domain, and a 41 BB intracellular domain. WU71A is a chimeric receptor with an anti-CD19 scFv extracellular domain, a CD16 transmembrane domain, and 2B4 and CD79A/B intracellular domains.

FIG. 13B provides graphs showing the median fluorescent intensity of cell surface CD3 expression by 293T-X cells transduced with lentivirus plasmid constructs encoding WU76E and WU71A. Expression was assessed by flow cytometry staining using 5 different anti-CD3 antibody clones: OKT3, UCHT1, TR66, HIT3a, or SK7.

FIG. 14 provides representative plots of flow cytometry staining of cell surface CD3 and CD56 on NK cells transduced with WU76E. Cells were analyzed on day 6 and day 14 of manufacturing.

FIG. 15 provides a graph showing the fold expansion of non-transduced NK cells (NK101), NK cells transduced with WU71A and NK cells transduced with WU76E over the course of 14 days in culture.

FIG. 16A provides representative plots of flow cytometry staining of cell surface CD56 and CD3 on three populations of cells: WU76E-NK cells prior to CD3 positive selection using STEMCELL human CD3 Positive Selective kits (Presort), WU76E-NK cells that were positively selected by the kit (Post Sort) and the flow through population that was not selected.

FIG. 16B provides representative plots of flow cytometry staining of cell surface CD56 and CD3 on WU76E-NK cells that were positively selected using STEMCELL human CD3 Positive Selection kits after 24 hours of culturing and on T cells.

FIG. 17 provides the Incucyte scan parameters used to quantify GFP+ NALM6 cancer cells and representative images of the phase and green signals captured by Incucyte, as well as illustrative images of the green object mask analysis performed by the Incucyte software to identify viable NALM6 cells.

FIG. 18 provides a graph showing the cell growth of NALM6 cells cultured in the presence of different concentrations of Blinatumomab. Cell growth was tracked using Incucyte and normalized to the starting number of cells.

FIG. 19 provides graphs showing the quantification of viable NALM6 cells remaining in each test condition over the course of 120 hours. NALM6 cells were co-cultured with different ratios of naïve T cells in the presence of 5 μg/mL, 312 ng/mL, 78 ng/mL or Ong/mL of Blinatumomab and growth of NALM6 cells were tracked via Incucyte and normalized to the number of starting cells.

FIG. 20 provides a graph showing the quantification of viable NALM6 cells remaining in each test condition over the course of 72 hours as tracked by Incucyte. Isolated NK cells were primed, expanded and transduced with lentivirus expressing WU76E and subsequently positively selected for CD3 on day 14 of culturing. The NALM6 cells and WU76E-NK cells were used on day 15 for cytotoxicity assays and tested under six different conditions:
(1) target only—NALM6 cells alone,
(2) target+Blina, NALM6 cells with 100 ng/mL of Blinatumomab,
(3) T Cell—NALM6 cells co-cultured with naïve, isolated CD3+ T cells at a 1:3 effector to target ratio in the presence of 100 ng/mL of Blinatumomab
(4) NK101—NALM6 cells co-cultured with NK101 at a 1:3 effector to target ratio in the presence of 100 ng/mL of Blinatumomab
(5) CAR19-NK—NALM6 cells co-cultured with WU71A-NK cells at a 1:3 effector to target ratio in the presence of 100 ng/mL of Blinatumomab
(6) CD3CAR-NK—NALM6 cells co-cultured with WU76E-NK cells at a 1:3 effector to target ratio in the presence of 100 ng/mL of Blinatumomab
   All T and NK cells are isolated from the same donor and the assay was carried out in the presence of 100 IU/mL of IL-2 in complete RPMI.

### DETAILED DESCRIPTION OF THE INVENTION

The present disclosure relates to NK cells modified to contain a chimeric antigen receptors (CAR) and methods of making and using the same.

### Chimeric Antigen Receptors (CARS)

The production and use of CARs is well established. CARs are generally designed in a modular fashion that includes at least an extracellular target-binding domain, a hinge region, a transmembrane domain that anchors the CAR to the cell membrane, and one or more intracellular signaling domains (also known as costimulatory domains) that transmit activation signals within the cell. The domains present within a CAR construct are operably linked with suitable linker sequences.

Much of the information known about CARs has come from studies of CAR T cells. More recently, however, CAR NK cells are beginning to show unique promise therapeutically speaking. Introduction of a functional CAR molecule into an NK cell can effectively redirect the NK cell with new antigen specificity and can provide the necessary signals to drive full NK cell activation. Also, because antigen recognition by CAR-modified NK cells is based on the binding of the target-binding region of an extracellular domain (e.g., an scFv sequence) to intact surface antigens, targeting of tumor cells is not MHC restricted, co-receptor dependent, or dependent on processing and effective presentation of target epitopes.

Some specific illustrative CAR domain sequences are set out below. It will be understood that the present disclosure is not limited to the use of these specific sequences. Rather, it will be understood that these specific sequences can be modified in various ways while still retaining a desired level of activity or functionality within an NK CAR. As such, in addition to the specific CAR domain sequences described below, the present disclosure also relates to functional fragments and variants of such sequences, e.g., nucleic acid sequences having at least 80%, 85%, 90%, 95% or 99% identity to a specific nucleic acid sequence disclosed herein, as well as to functional fragments and variants comprising amino acid sequences that are at least 80%, 85%, 90%, 95% or 99% identity to an amino acid sequence encoded by a specific CAR domain sequence disclosed herein.

A. CAR Intracellular Signaling Domains

The present invention is based, in part, on the identification of CAR intracellular signaling domains that are highly active in NK cells and offer additional unexpected advantages. More specifically, it has been found that the use of a combination of intracellular signaling domains selected or derived from CD79A, CD79B, 2B4 and/or DAP10 in NK CAR constructs overcomes many of the limitations associated with other intracellular signaling domains that have been used in NK cells by providing a more potent chimeric receptor compared to those commonly used in CAR T cells and also compared to those derived from endogenous NK cell receptors. As a result, NK cells containing the CAR constructs of the present disclosure are more effective at lower doses and more resilient to tumor immuno-suppression and evasion. In addition, transduction with CARs containing these intracellular signaling domains results in self-enrichment of transduced NK cells during manufacturing and the failure of contaminating T cells to expand as observed when using standard T cell intracellular signaling domains.

Therefore, in some embodiments, the present disclosure provides a chimeric antigen receptor (CAR) construct capable of being expressed in a natural killer (NK) cell, where the CAR construct comprises a combination of intracellular signaling domains selected or derived from a CD79A intracellular signaling domain, a CD79B intracellular signaling domain, a 2B4 intracellular signaling domain and a DAP10 signaling domain.

Some specific illustrative intracellular signaling domain sequences for these and other intracellular domains are set out below.

Illustrative Intracellular Signaling Domain Sequences:

CD79a_WT
(SEQ ID NO: 1)
CGCAAGCGCTGGCAGAACGAGAAGCTGGGCCTGGACGCCGGCGAC
GAGTACGAGGACGAGAACCTGTACGAGGGCCTGAACCTGGACGAC
TGCTCCATGTACGAGGACATCTCCCGCGGCCTGCAGGGCACCTAC
CAGGACGTGGGCTCCCTGAACATCGGCGACGTGCAGCTGGAGAAG
CCC
CD79a_Mut
(SEQ ID NO: 2)
CGCAAGCGCTGGCAGAACGAGAAGCTGGGCCTGGACGCCGGCGAC
GAGTACGAGGACGAGAACCTGTACGAGGGCCTGAACCTGGACGAC
TGCGCCATGTACGAGGACATCGCCCGCGGCCTGCAGGGCGTGTAC
CAGGACGTGGGCTCCCTGAACATCGGCGACGTGCAGCTGGAGAAG
CCC
CD79b_WT
(SEQ ID NO: 3)
CTGGACAAGGACGACTCCAAGGCCGGCATGGAGGAGGACCACACC
TACGAGGGCCTGGACATCGACCAGACCGCCACCTACGAGGACATC
GTGACCCTGCGCACCGGCGAGGTGAAGTGGTCCGTGGGCGAGCAC
CCCGGCCAGGAG
2B4
(SEQ ID NO: 4)
TGGCGCCGCAAGCGCAAGGAGAAGCAGTCCGAGACCTCCCCCAAG
GAGTTCCTGACCATCTACGAGGACGTGAAGGACCTGAAGACCCGC
CGCAACCACGAGCAGGAGCAGACCTTCCCCGGCGGCGGCTCCACC
ATCTACTCCATGATCCAGTCCCAGTCCTCCGCCCCCACCTCCCAG
GAGCCCGCCTACACCCTGTACTCCCTGATCCAGCCCTCCCGCAAG
TCCGGCTCCCGCAAGCGCAACCACTCCCCCTCCTTCAACTCCACC
ATCTACGAGGTGATCGGCAAGTCCCAGCCCAAGGCCCAGAACCCC
GCCCGCCTGTCCCGCAAGGAGCTGGAGAACTTCGACGTGTACTCC
DAP10
(SEQ ID NO: 5)
CTGTGCGCCCGCCCCCGCCGCTCCCCCGCCCAGGAGGACGGCAAG
GTGTACATCAACATGCCCGGCCGCGGC

In addition to the presence of two or three intracellular signaling domains chosen or derived from a 2B4 intracellular signaling domain, a CD79a intracellular signaling domain and a CD79b intracellular signaling domain, it will be understood that one or more additional intracellular signaling domains may be utilized in a CAR construct of the present disclosure. Illustratively, in some embodiments, such intracellular signaling domain sequences can be chosen or derived from an intracellular signaling domain of CD132, CD137/41BB (TRAF, NFkB), DNAM-1 (Y-motif), NKp80 (Y-motif), CRACC (CS1/SLAMF7)::ITSM, CD2 (Y-motifs, MAPK/Erk), CD27 (TRAF, NFkB), or integrins, a cytokine receptor associated with persistence, survival, or metabolism, such as IL-2/15Rbyc Jak1/3, STAT3/5, PI3K/mTOR, and MAPK/ERK; a cytokine receptor associated with activation, such as IL-18R::NFkB, a cytokine receptor associated with IFN-γ production, such as IL-12R::STAT4; a cytokine receptor associated with cytotoxicity or persistence, such as IL-21R::Jak3/Tyk2, or STAT3.

CD132
(SEQ ID NO: 6)
TTCTGGCTGGAACGGACGATGCCCCGAATTCCCACCCTGAAGAAC
CTAGAGGATCTTGTTACTGAATACCACGGGAACTTTTCGGCCTGG
AGTGGTGTGTCTAAGGGACTGGCTGAGAGTCTGCAGCCAGACTAC
AGTGAACGACTCTGCCTCGTCAGTGAGATTCCCCCAAAAGGAGGG
GCCCTTGGGGAGGGGCCTGGGGCCTCCCCATGCAACCAGCATAGC
CCCTACTGGGCCCCCCCATGTTACACCCTAAAGCCTGAAACC
CD137/41BB
(SEQ ID NO: 7)
aaacggggcagaaagaaactcctgtatatattcaaacaaccattt
atgagaccagtacaaactactcaagaggaagatggctgtagctgc
cgatttccagaagaagaagaaggaggatgtgaactg
DNAM-1
(SEQ ID NO: 8)
aaggagaaggagagagagaagagatctatttacagagtcctggga
tacacagaaggcacccaataactatagaagtcccatctctaccag
tcaacctaccaatcaatccatggatgatacaagagaggatattta
tgtcaactatccaaccttctctcgcagaccaaagactagagttta
ag
NKp80
(SEQ ID NO: 9)
tttctcagggagtattgctaaaatgccaaaaaggaagttgttcaa
atgccactcagtatgaggacactggagatctaaaagtgaataatg
gcacaagaagaaatataagtaataaggacctttgtgcttcgagat
ctgcagaccagacagtactatgccaatcagaatggctcaaatacc
aagggaagtgttattggttctctaatgagatgaaaagctggagtg
acagttatgtgtattgtttggaaagaaaatctcatctactaatca
tacatgaccaacttgaaatggcttttatacagaaaaacctaagac
aattaaactacgtatggattgggcttaactttacctccttgaaaa
tgacatggacttgggtggatggttctccaatagattcaaagatat
tcttcataaagggaccagctaaagaaaacagctgtgctgccatta
aggaaagcaaaattttctctgaaacctgcagcagtgttttcaaat
ggatttgtcagtattag
NTBA
(SEQ ID NO: 10)
attccctatctttgtctactcagcgaacacagggccccgcagagt
ccgcaaggaacctagagtatgtttcagtgtctccaacgaacaaca
ctgtgtatgcttcagtcactcattcaaacagggaaacagaaatct
ggacacctagagaaaatgatactatcacaatttactccacaatta
atcattccaaagagagtaaacccactttttccagggcaactgccc
ttgacaatgtcgtgtaa
CRACC
(SEQ ID NO: 11)
agtacattgaagagaagaagagagtggacatttgtcgggaaactc
ctaacatatgcccccattctggagagaacacagagtacgacacaa
tccctcacactaatagaacaatcctaaaggaagatccagcaaata
cggtttactccactgtggaaataccgaaaaag
CD2
(SEQ ID NO: 12)
aaaaggaaaaaacagaggagtcggagaaatgatgaggagctggag
acaagagcccacagagtagctactgaagaaaggggccggaagccc
caccaaattccagcttcaacccctcagaatccagcaacttcccaa
catcctcctccaccacctggtcatcgttcccaggcacctagtcat
cgtcccccgcctcctggacaccgtgttcagcaccagcctcagaag
aggcctcctgctccgtcgggcacacaagttcaccagcagaaaggc
ccgcccctccccagacctcgagttcagccaaaacctccccatggg
gcagcagaaaactcattgtccccttcctctaattaa
CD27
(SEQ ID NO: 13)
atccttgtgatcttctctggaatgttccttgttttcaccctggcc
ggggccctgttcctccatcaacgaaggaaatatagatcaaacaaa
ggagaaagtcctgtggagcctgcagagccttgtcgttacagctgc
cccagggaggaggagggcagcaccatccccatccaggaggattac
cgaaaaccggagcctgcctgctccccctga
Integrins
A. ITGB1
(SEQ ID NO: 14)
aagcttttaatgataattcatgacagaagggagtttgctaaattt
gaaaaggagaaaatgaatgccaaatgggacacgggtgaaaatcct
atttataagagtgccgtaacaactgtggtcaatccgaagtatgag
ggaaaatga
B. ITGB2
(SEQ ID NO: 15)
aaggctctgatccacctgagcgacctccgggagtacaggcgcttt
gagaaggagaagctcaagtcccagtggaacaatgataatcccctt
ttcaagagcgccaccacgacggtcatgaaccccaagtttgctgag
agttag
C. ITGB3
(SEQ ID NO: 16)
aaactcctcatcaccatccacgaccgaaaagaattcgctaaattt
gaggaagaacgcgccagagcaaaatgggacacagccaacaaccca
ctgtataaagaggccacgtctaccttcaccaatatcacgtaccgg
ggcacttaa
IL15RB
(SEQ ID NO: 17)
aactgcaggaacaccgggccatggctgaagaaggtcctgaagtgt
aacaccccagacccctcgaagttcttttcccagctgagctcagag
catggaggagacgtccagaagtggctctcttcgcccttcccctca
tcgtccttcagccctggcggcctggcacctgagatctcgccacta
gaagtgctggagagggacaaggtgacgcagctgctcctgcagcag
gacaaggtgcctgagcccgcatccttaagcagcaaccactcgctg
accagctgcttcaccaaccagggttacttcttcttccacctcccg
gatgccttggagatagaggcctgccaggtgtactttacttacgac
ccctactcagaggaagaccctgatgagggtgtggccggggcaccc
acagggtcttccccccaacccctgcagcctctgtcaggggaggac
gacgcctactgcaccttcccctccagggatgacctgctgctcttc
tcccccagtctcctcggtggccccagccccccaagcactgcccct
gggggcagtggggccggtgaagagaggatgcccccttctttgcaa
gaaagagtccccagagactgggacccccagcccctggggcctccc
accccaggagtcccagacctggtggattttcagccaccccctgag
ctggtgctgcgagaggctggggaggaggtccctgacgctggcccc
agggagggagtcagtttcccctggtccaggcctcctgggcagggg
gagttcagggcccttaatgctcgcctgcccctgaacactgatgcc
tacttgtccctccaagaactccagggtcaggacccaactcacttg
gtgtag
IL18R
(SEQ ID NO: 18)
tataaagttgacttggttctgttctataggcgcatagcggaaaga
gacgagacactaacagatggtaaaacatatgatgcctttgtgtct
tacctgaaagagtgtcatcctgagaataaagaagagtatactttt
gctgtggagacgttacccagggtcctggagaaacagtttgggtat
aagttatgcatatttgaaagagatgtggtgcctggcggagctgtt
gtcgaggagatccattcactgatagagaaaagccggaggctaatc
atcgttctcagccagagttacctgactaacggagccaggcgtgag
ctcgagagtggactccacgaagcactggtagagaggaagattaag
atcatcttaattgagtttactccagccagcaacatcacctttctc
cccccgtcgctgaaactcctgaagtcctacagagttctaaaatgg
agggctgacagtccctccatgaactcaaggttctggaagaatctt
gtttacctgatgcccgcaaaagccgtcaagccatggagagaggag
tcggaggcgcggtctgttctctcagcaccttga
IL12R
IL12RB1
(SEQ ID NO: 19)
aacagggccgcacggcacctgtgcccgccgctgcccacaccctgt
gccagctccgccattgagttccctggagggaaggagacttggcag
tggatcaacccagtggacttccaggaagaggcatccctgcaggag
gccctggtggtagagatgtcctgggacaaaggcgagaggactgag
cctctcgagaagacagagctacctgagggtgcccctgagctggcc
ctggatacagagttgtccttggaggatggagacaggtgcaaggcc
aagatgtga
IL12RB2
(SEQ ID NO: 20)
cattacttccagcaaaaggtgtttgttctcctagcagccctcaga
cctcagtggtgtagcagagaaattccagatccagcaaatagcact
tgcgctaagaaatatcccattgcagaggagaagacacagctgccc
ttggacaggctcctgatagactggcccacgcctgaagatcctgaa
ccgctggtcatcagtgaagtccttcatcaagtgaccccagttttc
agacatcccccctgctccaactggccacaaagggaaaaaggaatc
caaggtcatcaggcctctgagaaagacatgatgcacagtgcctca
agcccaccacctccaagagctctccaagctgagagcagacaactg
gtggatctgtacaaggtgctggagagcaggggctccgacccaaag
cccgaaaacccagcctgtccctggacggtgctcccagcaggtgac
cttcccacccatgatggctacttaccctccaacatagatgacctc
ccctcacatgaggcacctctcgctgactctctggaagaactggag
cctcagcacatctccctttctgttttcccctcaagttctcttcac
ccactcaccttctcctgtggtgataagctgactctggatcagtta
aagatgaggtgtgactccctcatgctctga
IL21R
(SEQ ID NO: 21)
agcctgaagacccatccattgtggaggctatggaagaagatatgg
gccgtccccagccctgagcggttcttcatgcccctgtacaagggc
tgcagcggagacttcaagaaatgggtgggtgcacccttcactggc
tccagcctggagctgggaccctggagcccagaggtgccctccacc
ctggaggtgtacagctgccacccaccacggagcccggccaagagg
ctgcagctcacggagctacaagaaccagcagagctggtggagtct
gacggtgtgcccaagcccagcttctggccgacagcccagaactcg
gggggctcagcttacagtgaggagagggatcggccatacggcctg
gtgtccattgacacagtgactgtgctagatgcagaggggccatgc
acctggccctgcagctgtgaggatgacggctacccagccctggac
ctggatgctggcctggagcccagcccaggcctagaggacccactc
ttggatgcagggaccacagtcctgtcctgtggctgtgtctcagct
ggcagccctgggctaggagggcccctgggaagcctcctggacaga
ctaaagccaccccttgcagatggggaggactgggctgggggactg
ccctggggtggccggtcacctggaggggtctcagagagtgaggcg
ggctcacccctggccggcctggatatggacacgtttgacagtggc
tttgtgggctctgactgcagcagccctgtggagtgtgacttcacc
agccccggggacgaaggacccccccggagctacctccgccagtgg
gtggtcattcctccgccactttcgagccctggaccccaggccagc
taa
IRE1a
(SEQ ID NO: 22)
cccctgagcatgcatcagcagcagcagctccagcaccagcagttc
cagaaggaactggagaagatccagctcctgcagcagcagcagcag
cagctgcccttccacccacctggagacacggctcaggacggcgag
ctcctggacacgtctggcccgtactcagagagctcgggcaccagc
agccccagcacgtcccccagggcctccaaccactcgctctgctcc
ggcagctctgcctccaaggctggcagcagcccctccctggaacaa
gacgatggagatgaggaaaccagcgtggtgatagttgggaaaatt
tccttctgtcccaaggatgtcctgggccatggagctgagggcaca
attgtgtaccggggcatgtttgacaaccgcgacgtggccgtgaag
aggatcctccccgagtgttttagcttcgcagaccgtgaggtccag
ctgttgcgagaatcggatgagcacccgaacgtgatccgctacttc
tgcacggagaaggaccggcaattccagtacattgccatcgagctg
tgtgcagccaccctgcaagagtatgtggagcagaaggactttgcg
catctcggcctggagcccatcaccttgctgcagcagaccacctcg
ggcctggcccacctccactccctcaacatcgttcacagagaccta
aagccacacaacatcctcatatccatgcccaatgcacacggcaag
atcaaggccatgatctccgactttggcctctgcaagaagctggca
gtgggcagacacagtttcagccgccgatctggggtgcctggcaca
gaaggctggatcgctccagagatgctgagcgaagactgtaaggag
aaccctacctacacggtggacatcttttctgcaggctgcgtcttt
tactacgtaatctctgagggcagccacccttttggcaagtccctg
cagcggcaggccaacatcctcctgggtgcctgcagccttgactgc
ttgcacccagagaagcacgaagacgtcattgcacgtgaattgata
gagaagatgattgcgatggatcctcagaaacgcccctcagcgaag
catgtgctcaaacacccgttcttctggagcctagagaagcagctc
cagttcttccaggacgtgagcgacagaatagaaaaggaatccctg
gatggcccgatcgtgaagcagttagagagaggcgggagagccgtg
gtgaagatggactggcgggagaacatcactgtccccctccagaca
gacctgcgtaaattcaggacctataaaggtggttctgtcagagat
ctcctccgagccatgagaaataagaagcaccactaccgggagctg
cctgcagaggtgcgggagacgctggggtccctccccgacgacttc
gtgtgctacttcacatctcgcttcccccacctcctcgcacacacc
taccgggccatggagctgtgcagccacgagagactcttccagccc
tactacttccacgagcccccagagccccagcccccagtgactcca
gacgccctctga

B. CAR Extracellular Domains

The CAR constructs of the present disclosure generally include an extracellular domain. In some embodiments, the extracellular domains is capable of binding to a target polypeptide of interest, such as an antigen associated with an infectious disease, a bacterial infection, a virus, a cancer, an autoimmune disease, or an immune disorder or dysfunction.

In some embodiments, the extracellular domain of a CAR construct of the present disclosure comprises an antibody fragment. For example, in some embodiments, the extracellular domain of a CAR construct of the present disclosure comprises a single-chain variable fragment sequence (scFv sequence) capable of binding a target polypeptide of interest, such as a disease-associated target polypeptide.

scFvs are well known in the art to be used as a binding moiety in a variety of constructs (see e.g., Sentman 2014 Cancer J. 20 156-159; Guedan 2019 Mol Ther Methods Clin Dev. 12 145-156). scFvs can be against any antigen known in the art, such as those described in US20160361360A1, which is incorporated herein by reference in its entirety. Any scFv known in the art or generated against an antigen using means known in the art can be used as the binding moiety in an extracellular domain of a CAR construct of the present disclosure.

The format of a scFv is generally two variable domains linked by a flexible peptide sequence, either in the orientation VH-linker-VL or VL-linker-VH. The orientation of the variable domains within the scFv, depending on the structure of the scFv, may contribute to whether a CAR will be expressed on the NK cell surface or whether the NK cells target the antigen and signal. In addition, the length and/or composition of the variable domain linker can contribute to the stability or affinity of the scFv.

CAR scFv affinities, modified through mutagenesis of complementary-determining regions while holding the epitope constant, or through CAR development with scFvs derived from therapeutic antibodies against the same target, but not the same epitope, can change the strength of the NK cell signal and allow NK cells to differentiate overexpressed antigens from normally expressed antigens. The scFv, a critical component of a CAR molecule, can be carefully designed and manipulated to influence specificity and differential targeting of tumors versus normal tissues. Given that these differences may only be measurable with NK cells (as opposed to soluble antibodies), pre-clinical testing of normal tissues for expression of the target, and susceptibility to on-target toxicities, requires live-cell assays rather than immunohistochemistry on fixed tissues.

In some embodiments, the scFvs described herein can be used for hematological malignancies such as AML, ALL, or Lymphoma, but can also be expanded for use in any malignancy, autoimmune, or infectious disease where a scFv can be generated against a target antigen or antigen epitope. For example, the constructs described herein can be used to treat or prevent autoimmunity associated with auto-antibodies (similar indications as rituximab for autoimmunity). As another example, the disclosed constructs can also be applied to virally infected cells, using scFv that can recognize viral antigens, for example gp120 and gp41 on HIV-infected cells.

Examples of disease-associated polypeptides that can be advantageously targeted by the CAR extracellular domains according to the disclosure can include essentially any known target. In some embodiments, the polypeptide targeted and bound by the extracellular domain of the CAR is selected from the group consisting of CD2, CD5, CD7, MSLN, CEA, PSMA, CD19, CD28, CD3, CD33, CD38, CD138, CLL-1, CLL-3, C-KIT CD123, CD133, CD20, BCMA, EGFR, CD3, CD4, BAFF-R, EGFR, HER2, GD2, gp120 and gp41.

Illustrative scFv sequences capable of binding some of these target polypeptides are provided below.

Anti-Mesothelin scFv
(SEQ ID NO: 23)
ATGGCCTTACCAGTGACCGCCTTGCTCCTGCCGCTGGCCTTGCTG
CTCCACGCCGCCAGGCCGGGATCCCAGGTACAACTGCAGCAGTCT
GGGCCTGAGCTGGAGAAGCCTGGCGCTTCAGTGAAGATATCCTGC
AAGGCTTCTGGTTACTCATTCACTGGCTACACCATGAACTGGGTG
AAGCAGAGCCATGGAAAGAGCCTTGAGTGGATTGGACTTATTACT
CCTTACAATGGTGCTTCTAGCTACAACCAGAAGTTCAGGGGCAAG
GCCACATTAACTGTAGACAAGTCATCCAGCACAGCCTACATGGAC
CTCCTCAGTCTGACATCTGAAGACTCTGCAGTCTATTTCTGTGCA
AGGGGGGGTTACGACGGGAGGGGTTTTGACTACTGGGGCCAAGGG
ACCACGGTCACCGTCTCCTCAGGTGGAGGCGGTTCAGGCGGCGGT
GGCTCTAGCGGTGGTGGATCGGACATCGAGCTCACTCAGTCTCCA
GCAATCATGTCTGCATCTCCAGGGGAGAAGGTCACCATGACCTGC
AGTGCCAGCTCAAGTGTAAGTTACATGCACTGGTACCAGCAGAAG
TCAGGCACCTCCCCCAAAAGATGGATTTATGACACATCCAAACTG
GCTTCTGGAGTCCCAGGTCGCTTCAGTGGCAGTGGGTCTGGAAAC
TCTTACTCTCTCACAATCAGCAGCGTGGAGGCTGAAGATGATGCA
ACTTATTACTGCCAGCAGTGGAGTAAGCACCCTCTCACGTACGGT
GCTGGGACAAAGTTGGAAATCAAAGCTAGC
Anti-CD19 scFv
(SEQ ID NO: 24)
ATGGCCTTACCAGTGACCGCCTTGCTCCTGCCGCTGGCCTTGCTG
CTCCACGCCGCCAGGCCGGACATCCAGATGACACAAACCACCTCC
AGCCTGTCCGCATCCCTTGGAGATCGAGTCACCATTAGCTGCCGG
GCGTCCCAGGACATCAGCAAATACCTCAATTGGTATCAGCAAAAA
CCCGACGGCACGGTGAAGCTTCTGATATACCACACTTCACGGCTG
CACTCAGGCGTGCCAAGTCGGTTTTCAGGATCCGGCTCCGGCACC
GATTATTCTCTGACCATTAGCAATCTCGAGCAAGAGGATATCGCC
ACTTATTTCTGTCAGCAGGGGAACACCCTCCCTTATACGTTTGGT
GGTGGGACCAAGTTGGAGATTACACGGGCAGATGCCGCCCCTACT
GTGAGCATATTTCCTCCTAGCTCTAATGGGGGTGGAGGCTCCGGT
GGTGGGGGGAGCGGCGGCGGTGGCTCAGGAGGCGGCGGCAGCGAG
GTCAAACTGCAAGAGTCTGGTCCAGGCCTGGTGGCCCCCTCCCAG
TCTCTCAGTGTGACCTGTACCGTTTCAGGCGTGTCTTTGCCTGAT
TACGGCGTGTCCTGGATAAGGCAGCCACCAAGAAAGGGACTGGAA
TGGCTGGGCGTTATCTGGGGATCAGAGACAACTTATTACAACTCA
GCTCTTAAATCCAGACTCACGATCATTAAGGATAACTCTAAATCC
CAGGTGTTCCTGAAGATGAATTCTCTCCAGACAGACGATACTGCC
ATCTACTATTGCGCTAAGCATTATTATTACGGGGGCTCATACGCC
ATGGACTACTGGGGACAGGGCACCAGCGTGACTGTGAGTTCCCCT
AGGGCTAGC
Anti-CD19 scFv
(SEQ ID NO: 25)
ATGGCCCTGCCCGTGACCGCTCTCCTGCTGCCTCTGGCCCTGCTC
CTCCATGCTGCCAGACCCGACATCCAGATGACACAGACAACCAGC
AGCCTGTCCGCTTCCCTCGGAGACAGGGTGACAATTTCCTGCAGG
GCCAGCCAGGACATCAGCAAGTACCTGAACTGGTACCAGCAGAAA
CCCGACGGCACCGTCAAGCTCCTGATCTACCACACCAGCAGACTG
CACAGCGGAGTGCCTTCCAGGTTCAGCGGCAGCGGCTCCGGCACC
GATTACTCCCTGACCATTAGCAACTTAGAACAGGAGGACATTGCC
ACCTACTTTTGTCAGCAGGGCAACACCCTCCCCTACACCTTTGGA
GGCGGAACCAAGTTAGAAATCACCGGCGGCGGCGGCAGCGGAGGA
GGAGGCAGCGGAGGCGGAGGCTCCGAGGTGAAACTGCAGGAGAGC
GGCCCCGGACTGGTCGCCCCTAGCCAATCCCTCTCCGTCACCTGC
ACCGTGAGCGGAGTGAGCCTGCCTGACTACGGAGTGAGCTGGATC
AGACAGCCCCCTAGGAAAGGACTGGAATGGCTGGGCGTGATTTGG
GGCAGCGAGACCACCTATTACAACAGCGCCCTGAAGTCCAGACTG
ACAATCATCAAGGACAATAGCAAAAGCCAAGTGTTTCTGAAGATG
AACAGCCTGCAGACCGATGACACCGCCATCTATTATTGCGCCAAG
CACTACTACTACGGAGGAAGCTACGCTATGGATTATTGGGGCCAA
GGCACAAGCGTGACCGTCAGCAGCGCGGCCGCC
Anti-CD33 scFv
(SEQ ID NO: 26)
ATGGCCTTACCAGTGACCGCCTTGCTCCTGCCGCTGGCCTTGCTG
CTCCACGCCGCCAGGCCGATGGAAAAGGATACACTGTTGTTGTGG
GTTCTGCTCCTGTGGGTGCCCGGCAGCACCGGAGATATTGTGCTG
ACGCAGTCTCCTGCATCACTCGCCGTGTCTCTGGGCCAGCGCGCT
ACCATCAGCTGCAGAGCCTCTGAAAGTGTTGACAATTATGGAATT
TCTTTCATGAATTGGTTCCAGCAGAAGCCTGGCCAGCCCCCGAAA
CTCCTCATATATGCCGCGTCTAATCAGGGCTCTGGGGTCCCTGCT
AGATTTTCTGGCAGCGGCTCCGGCACCGACTTCAGTCTGAATATA
CATCCCATGGAAGAAGACGATACCGCCATGTACTTTTGCCAACAA
TCTAAGGAGGTGCCTTGGACGTTCGGCGGCGGTACGAAGCTGGAA
ATTAAGGGGGGGGGGGGAAGCGGCGGGGGGGGATCAGGCGGGGGT
GGCTCCGGAGGCGGTGGAAGTATGGGCTGGAGTTGGATCTTCCTT
TTCCTTCTTTCTGGTACCGCGGGAGTGCACTCTGAGGTGCAGCTC
CAGCAGTCCGGCCCCGAGCTCGTCAAGCCTGGGGCCAGTGTCAAG
ATTTCCTGTAAGGCATCTGGATATACCTTTACAGATTACAATATG
CATTGGGTGAAACAGTCACATGGAAAGTCACTCGAGTGGATCGGA
TACATTTACCCTTACAATGGAGGAACCGGATATAATCAGAAGTTT
AAGAGCAAGGCCACACTCACGGTGGACAATTCTTCATCTACAGCC
TACATGGATGTTCGGTCTCTGACTTCCGAGGATAGTGCGGTGTAT
TACTGCGCCAGGGGACGCCCCGCTATGGATTACTGGGGGCAGGGA
ACCTCTGTAACAGTTAGCTCA
Anti-CD123 scFv
(SEQ ID NO: 27)
ATGGCCTTACCAGTGACCGCCTTGCTCCTGCCGCTGGCCTTGCTG
CTCCACGCCGCCAGGCCGGACTTCGTGATGACTCAGTCTCCTAGC
TCCCTGACCGTGACAGCCGGCGAGAAGGTGACCATGTCCTGCAAA
TCTAGTCAGAGTCTGCTGAACTCAGGCAATCAGAAGAACTATCTG
ACATGGTACCTGCAGAAGCCAGGGCAGCCCCCTAAACTGCTGATC
TATTGGGCCAGCACCAGGGAATCCGGCGTGCCCGACAGATTCACC
GGCTCCGGGTCTGGAACAGATTTTACTCTGACCATTTCAAGCGTG
CAGGCCGAGGACCTGGCTGTGTACTATTGTCAGAATGATTACAGC
TATCCCTACACATTTGGCGGGGGAACTAAGCTGGAAATCAAAGGT
GGTGGTGGTTCTGGTGGTGGTGGTTCCGGCGGCGGCGGCTCCGGT
GGTGGTGGATCCGAGGTGCAGCTGCAGCAGAGTGGACCCGAACTG
GTGAAACCTGGCGCCTCCGTGAAAATGTCTTGCAAGGCTAGTGGG
TACACCTTCACAGACTACTATATGAAATGGGTGAAGCAGTCACAC
GGGAAGAGCCTGGAGTGGATCGGAGATATCATTCCCTCTAACGGC
GCCACTTTCTACAATCAGAAGTTTAAAGGCAAGGCTACTCTGACC
GTGGACCGGAGCTCCTCTACCGCCTATATGCACCTGAACAGTCTG
ACATCAGAAGATAGCGCTGTGTACTATTGTACACGGTCCCATCTG
CTGAGAGCCTCTTGGTTTGCTTATTGGGGCCAGGGGACACTGGTG
ACTGTGAGCTCCGCTAGCACCACGACGCCAGCGCCGCGACCACCA
ACACCGGCGCCCACCATCGCGTCGCAGCCCCTGTCCCTGCGCCCA
GAGGCGTGCCGGCCAGCGGCGGGGGGCGCAGTGCACACGAGGGGG
CTGGACTTCGCCTGTGATTTTTGGGTGCTGGTGGTGGTTGGTGGA
GTCCTGGCTTGCTATAGCTTGCTAGTAACAGTGGCCTTTATTATT
TTCTGGGTG

Other CAR Extracellular Domains

In some embodiments of the present disclosure, the extracellular domain of a CAR is one that is derived from a cellular receptor, such as an Fc receptor, such as CD16, CD32, CD64 or others. Extracellular domains comprising such sequences are particularly useful in the production of ADCC-enabled NK cells containing a CAR construct of the disclosure. In some embodiments, the CD16 sequence has a mutation corresponding to S197P at the ADAM17 cleavage site in order inhibit cleavage and shedding of the expressed CAR to enhance NK cell activity. As described herein, such Enhanced (E)-ADCC-enabled NK cells exhibit increased cytotoxicity towards different cancer cell types when compared to control NK cells.

Additionally, similarly to the other CAR constructs encoding or expressing the intracellular signaling domains described herein, self-enrichment of the CAR-expressing NK cells was observed during the manufacturing process.

Illustrative CD16, CD32 and CD64 sequences capable of being used in these ways are set out below.

CD16_S197P ECD-w/mutation at ADAM17 cleavage site
(SEQ ID NO: 28)
ATGGCCTTACCAGTGACCGCCTTGCTCCTGCCGCTGGCCTTGCTG
CTCCACGCCGCCAGGCCGGGCATGCGCACCGAGGACCTGCCCAAG
GCCGTGGTGTTCCTGGAGCCCCAGTGGTACCGCGTGCTGGAGAAG
GACTCCGTGACCCTGAAGTGCCAGGGCGCCTACTCCCCCGAGGAC
AACTCCACCCAGTGGTTCCACAACGAGTCCCTGATCTCCTCCCAG
GCCTCCTCCTACTTCATCGACGCCGCCACCGTGGACGACTCCGGC
GAGTACCGCTGCCAGACCAACCTGTCCACCCTGTCCGACCCCGTG
CAGCTGGAGGTGCACATCGGCTGGCTGCTGCTGCAGGCCCCCCGC
TGGGTGTTCAAGGAGGAGGACCCCATCCACCTGCGCTGCCACTCC
TGGAAGAACACCGCCCTGCACAAGGTGACCTACCTGCAGAACGGC
AAGGGCCGCAAGTACTTCCACCACAACTCCGACTTCTACATCCCC
AAGGCCACCCTGAAGGACTCCGGCTCCTACTTCTGCCGCGGCCTG
TTCGGCTCCAAGAACGTGTCCTCCGAGACCGTGAACATCACCATC
ACCCAGGGCCTGGCCGTGCCCACCATCTCCTCCTTCTTCCCCCCC
GGCTACCAG
CD32 ECD
(SEQ ID NO: 29)
ATGGCCTTACCAGTGACCGCCTTGCTCCTGCCGCTGGCCTTGCTG
CTCCACGCCGCCAGGCCGCAGGCCGCCGCCCCCCCCAAGGCCGTG
CTGAAGCTGGAGCCCCCCTGGATCAACGTGCTGCAGGAGGACTCC
GTGACCCTGACCTGCCAGGGCGCCCGCTCCCCCGAGTCCGACTCC
ATCCAGTGGTTCCACAACGGCAACCTGATCCCCACCCACACCCAG
CCCTCCTACCGCTTCAAGGCCAACAACAACGACTCCGGCGAGTAC
ACCTGCCAGACCGGCCAGACCTCCCTGTCCGACCCCGTGCACCTG
ACCGTGCTGTCCGAGTGGCTGGTGCTGCAGACCCCCCACCTGGAG
TTCCAGGAGGGCGAGACCATCATGCTGCGCTGCCACTCCTGGAAG
GACAAGCCCCTGGTGAAGGTGACCTTCTTCCAGAACGGCAAGTCC
CAGAAGTTCTCCCACCTGGACCCCACCTTCTCCATCCCCCAGGCC
AACCACTCCCACTCCGGCGACTACCACTGCACCGGCAACATCGGC
TACACCCTGTTCTCCTCCAAGCCCGTGACCATCACCGTGCAGGTG
CCCTCCATGGGCTCCTCCTCCCCCATGGGC
CD64 ECD
(SEQ ID NO: 30)
ATGGCCTTACCAGTGACCGCCTTGCTCCTGCCGCTGGCCTTGCTG
CTCCACGCCGCCAGGCCGCAGGTGGACACCACCAAGGCCGTGATC
ACCCTGCAGCCCCCCTGGGTGTCCGTGTTCCAGGAGGAGACCGTG
ACCCTGCACTGCGAGGTGCTGCACCTGCCCGGCTCCTCCTCCACC
CAGTGGTTCCTGAACGGCACCGCCACCCAGACCTCCACCCCCTCC
TACCGCATCACCTCCGCCTCCGTGAACGACTCCGGCGAGTACCGC
TGCCAGCGCGGCCTGTCCGGCCGCTCCGACCCCATCCAGCTGGAG
ATCCACCGCGGCTGGCTGCTGCTGCAGGTGTCCTCCCGCGTGTTC
ACCGAGGGCGAGCCCCTGGCCCTGCGCTGCCACGCCTGGAAGGAC
AAGCTGGTGTACAACGTGCTGTACTACCGCAACGGCAAGGCCTTC
AAGTTCTTCCACTGGAACTCCAACCTGACCATCCTGAAGACCAAC
ATCTCCCACAACGGCACCTACCACTGCTCCGGCATGGGCAAGCAC
CGCTACACCTCCGCCGGCATCTCCGTGACCGTGAAGGAGCTGTTC
CCCGCCCCCGTGCTGAACGCCTCCGTGACCTCCCCCCTGCTGGAG
GGCAACCTGGTGACCCTGTCCTGCGAGACCAAGCTGCTGCTGCAG
CGCCCCGGCCTGCAGCTGTACTTCTCCTTCTACATGGGCTCCAAG
ACCCTGCGCGGCCGCAACACCTCCTCCGAGTACCAGATCCTGACC
GCCCGCCGCGAGGACTCCGGCCTGTACTGGTGCGAGGCCGCCACC
GAGGACGGCAACGTGCTGAAGCGCTCCCCCGAGCTGGAGCTGCAG
GTGCTGGGCCTGCAGCTGCCCACCCCCGTGTGGTTCCAC

In other embodiments of the CARs of the disclosure, the extracellular domain that is present in the CAR comprises an extracellular domain of CD3e or CD28. The CD3e and CD28 ECDs can be used, for example, in combination with bispecific clinical antibodies such as blinatumomab where one Fab targets CD3 and the other Fab targets CD19.

CD3e ECD
(SEQ ID NO: 31)
ATGGCCTTACCAGTGACCGCCTTGCTCCTGCCGCTGGCCTTGCTG
CTCCACGCCGCCAGGCCGGACGGCAACGAGGAGATGGGCGGCATC
ACCCAGACCCCCTACAAGGTGTCCATCTCCGGCACCACCGTGATC
CTGACCTGCCCCCAGTACCCCGGCTCCGAGATCCTGTGGCAGCAC
AACGACAAGAACATCGGCGGCGACGAGGACGACAAGAACATCGGC
TCCGACGAGGACCACCTGTCCCTGAAGGAGTTCTCCGAGCTGGAG
CAGTCCGGCTACTACGTGTGCTACCCCCGCGGCTCCAAGCCCGAG
GACGCCAACTTCTACCTGTACCTGCGCGCCCGCGTGTGCGAGAAC
TGCATGGAGATGGAC
CD28 ECD
(SEQ ID NO: 32)
ATGGCCTTACCAGTGACCGCCTTGCTCCTGCCGCTGGCCTTGCTG
CTCCACGCCGCCAGGCCGAACAAGATCCTGGTGAAGCAGTCCCCC
ATGCTGGTGGCCTACGACAACGCCGTGAACCTGTCCTGCAAGTAC
TCCTACAACCTGTTCTCCCGCGAGTTCCGCGCCTCCCTGCACAAG
GGCCTGGACTCCGCCGTGGAGGTGTGCGTGGTGTACGGCAACTAC
TCCCAGCAGCTGCAGGTGTACTCCAAGACCGGCTTCAACTGCGAC
GGCAAGCTGGGCAACGAGTCCGTGACCTTCTACCTGCAGAACCTG
TACGTGAACCAGACCGACATCTACTTCTGCAAGATCGAGGTGATG
TACCCCCCCCCCTACCTGGACAACGAGAAGTCCAACGGCACCATC
ATCCACGTGAAGGGCAAGCACCTGTGCCCCTCCCCCCTGTTCCCC
GGCCCCTCCAAGCCCTTC

C. CAR Transmembrane (Tm) Domains and Adapters

The CAR constructs described herein also include a transmembrane (TM) domain consisting of a hydrophobic a helix that spans the cell membrane. Although the main function of the transmembrane is to anchor the CAR in the NK cell membrane, some evidence suggests that the transmembrane domain can be relevant for CAR cell function.

The TM domain can be any TM domain suitable that is effective in an NK cell. For example, in some embodiments, the TM domain can be chosen or derived from a transmembrane sequence from CD28, CD16, CD32, CD64, NKG2D, FcγRIIIa, NKp44, NKp30, NKp46, actKIR, NKG2C, CD8a or IL-15. NK cells express transmembrane (TM) adapters (e.g. DAP10, CD3z) that transmit activation signals upon association with activating receptors (e.g. CD16, NKG2D). This provides an NK cell-specific signal enhancement via engineering the TM domains from activating receptors, and thereby harness endogenous adapters. The TM adapter can be any endogenous TM adapter capable of signaling activation. For example, the TM adapter can be FceR1γ (ITAMx1), CD3ζ (ITAMx3), DAP12 (ITAMx1), or DAP10 (YxxM/YINM).

In certain embodiments, the TM domains and adapters may be paired, e.g.: NKG2D and DAP10, FcγRIIIa and CD3 or FceR1γ, NKp44 and DAP12, NKp30 and CD3 or FceR1γ, NKp46 and CD3 or FceR1γ, actKIR and DAP12, and NKG2C and DAP12.

D. Hinge (Spacer) Domains

The hinge, also referred to as a spacer, is in the extracellular structural region of the CAR that separates the binding units from the transmembrane domain. The hinge can be any moiety capable of ensuring proximity of the CAR NK cell to the target (e.g., NKG2-based hinge, TMα-based hinge, CD8-based hinge). With the exception of a few CARs based on the entire extracellular moiety of a receptor, such as NKG2D, as described herein, the majority of CAR (such as CAR T) cells are designed with immunoglobulin (1g)-like domain hinges.

Hinges generally supply stability for efficient CAR expression and activity. The NKG2 hinge (also in combination with the transmembrane domain), described herein also ensures proper proximity to target.

The hinge also provides flexibility to access the targeted antigen. The optimal spacer length of a given CAR can depend on the position of the targeted epitope. Long spacers can provide extra flexibility to the CAR and allow for better access to membrane-proximal epitopes or complex glycosylated antigens. CARs bearing short hinges can be more effective at binding membrane-distal epitopes. The length of the spacer can be important to provide adequate intercellular distance for immunological synapse formation. As such, hinges may be optimized for individual epitopes accordingly.

Below are illustrative hinge and TM domain sequences. It will be understood that the hinge and TM sequences described below can be mixed, matched and altered for optimal performance.

### Hinge/Transmembrane (TM) Domain Sequences

NKG2D
(Hinge (SEQ ID NO: 34)/TM (SEQ ID NO: 35))
TCCACAAGAATCAAGATCTTCCCTCTCTGAGCAGGAATCCTTTGT
GCATTGAAGACTTTAGATTCCTCTCTGCGGTAGACGTGCACTTAT
AAGTATTTGATGGGGTGGATTCGTGGTCGGAGGTCTCGACACAGC
TGGGAGATGAGTGAATTTCATAATTATAACTTGGATCTGAAGAAG
AGTGATTTTTCAACACGATGGCAAAAGCAAAGATGTCCAGTAGTC
AAAAGCAAATGTAGAGAAAATGCATCT/CCATTTTTTTTCTGCTG
CTTCATCGCTGTAGCCATGGGAATCCGTTTCATTATTATGGTAAC
AATATGGAGT
FcγRIIIa
(Hinge (SEQ ID NO: 36)/TM (SEQ ID NO: 37))
CACCTGAGGTGTCACAGCTGGAAGAACACTGCTCTGCATAAGGTC
ACATATTTACAGAATGGCAAAGGCAGGAAGTATTTTCATCATAAT
TCTGACTTCTACATTCCAAAAGCCACACTCAAAGACAGCGGCTCC
TACTTCTGCAGGGGGCTTTTTGGGAGTAAAAATGTGTCTTCAGAG
ACTGTGAACATCACCATCACTCAAGGTTTGGCAGTGTCAACCATC
TCATCATTCTTTCCACCTGGGTACCAA/GTCTCTTTCTGCTTGGT
GATGGTACTCCTTTTTGCAGTGGACACAGGACTATATTTCTCTGT
GAAGACAAACA
NKp44
(Hinge (SEQ ID NO: 38)/TM (SEQ ID NO: 39))
TGTAGAATCTACCGCCCTTCTGACAACTCTGTCTCTAAGTCCGTC
AGATTCTATCTGGTGGTATCTCCAGCCTCTGCCTCCACACAGACC
TCCTGGACTCCCCGCGACCTGGTCTCTTCACAGACCCAGACCCAG
AGCTGTGTGCCTCCCACTGCAGGAGCCAGACAAGCCCCTGAGTCT
CCATCTACCATCCCTGTCCCTTCACAGCCACAGAACTCCACGCTC
CGCCCTGGCCCTGCAGCCCCCATTGCC/CTGGTGCCTGTGTTCTG
TGGACTCCTCGTAGCCAAGAGCCTGGTGCTGTCAGCCCTGCTCGT
CTGGTGGGGG
NKp30
(Hinge (SEQ ID NO: 40)/TM (SEQ ID NO: 41))
TCCGTCACGTGGTTCCGAGATGAGGTGGTTCCAGGGAAGGAGGTG
AGGAATGGAACCCCAGAGTTCAGGGGCCGCCTGGCCCCACTTGCT
TCTTCCCGTTTCCTCCATGACCACCAGGCTGAGCTGCACATCCGG
GACGTGCGAGGCCATGACGCCAGCATCTACGTGTGCAGAGTGGAG
GTGCTGGGCCTTGGTGTCGGGACAGGGAATGGGACTCGGCTGGTG
GTGGAGAAAGAACATCCTCAGCTAGGG/GCTGGTACAGTCCTCCT
CCTTCGGGCTGGATTCTATGCTGTCAGCTTTCTCTCTGTGGCCGT
GGGCAGCACC
NKp46
(Hinge (SEQ ID NO: 42)/TM (SEQ ID NO: 43))
TTCCCCCTGGGCCCTGTGACCACAGCCCACAGAGGGACATACCGA
TGTTTTGGCTCCTATAACAACCATGCCTGGTCTTTCCCCAGTGAG
CCAGTGAAGCTCCTGGTCACAGGCGACATTGAGAACACCAGCCTT
GCACCTGAAGACCCCACCTTTCCTGCAGACACTTGGGGCACCTAC
CTTTTAACCACAGAGACGGGACTCCAGAAAGACCATGCCCTCTGG
GATCACACTGCCCAGAATCTCCTTCGG/ATGGGCCTGGCCTTTCT
AGTCCTGGTGGCTCTAGTGTGGTTCCTGGTTGAAGACTGGCTCAG
CAGGAAGAGG
Activating KIR (KIR2DS4)
(Hinge (SEQ ID NO: 44)/TM (SEQ ID NO: 45))
AGGGAAGGGGAGGCCCATGAACGTAGGCTCCCTGCAGTGCGCAGC
ATCAACGGAACATTCCAGGCCGACTTTCCTCTGGGCCCTGCCACC
CACGGAGGGACCTACAGATGCTTCGGCTCTTTCCGTGACGCTCCC
TACGAGTGGTCAAACTCGAGTGATCCACTGCTTGTTTCCGTCACA
GGAAACCCTTCAAATAGTTGGCCTTCACCCACTGAACCAAGCTCC
AAAACCGGTAACCCCAGACACCTACAT/GTTCTGATTGGGACCTC
AGTGGTCAAAATCCCTTTCACCATCCTCCTCTTCTTTCTCCTTCA
TCGCTGG
NKG2C
(Hinge (SEQ ID NO: 46)/TM (SEQ ID NO: 47))
ATGAATAAACAAAGAGGAACCTTCTCAGAAGTGAGTCTGGCCCAG
GACCCAAAGCGGCAGCAAAGGAAACCTAAAGGCAATAAAAGCTCC
ATTTCAGGAACCGAACAGGAAATATTCCAAGTAGAATTAAATCTT
CAAAATCCTTCCCTGAATCATCAAGGGATTGATAAAATATATGAC
TGCCAAGGTTTACTGCCACCTCCAGAGAAG/CTCACTGCCGAGGT
CCTAGGAATCATTTGCATTGTCCTGATGGCCACTGTGTTAAAAAC
AATAGTTCTTATTCCTTTC
CD8a
(Hinge (SEQ ID NO: 48)/TM (SEQ ID NO: 49))
GTCCTCACCCTGAGCGACTTCCGCCGAGAGAACGAGGGCTACTAT
TTCTGCTCGGCCCTGAGCAACTCCATCATGTACTTCAGCCACTTC
GTGCCGGTCTTCCTGCCAGCGAAGCCCACCACGACGCCAGCGCCG
CGACCACCAACACCGGCGCCCACCATCGCGTCGCAGCCCCTGTCC
CTGCGCCCAGAGGCGTGCCGGCCAGCGGGGGGGGCGCAGTGCACA
CGAGGGGGCTGGACTTCGCCTGTGAT/ATCTACATCTGGGCGCCC
TTGGCCGGGACTTGTGGGGTCCTTCTCCTGTCACTGGTTATCACC
CTTTACTGC
IL15Rb
(Hinge (SEQ ID NO: 50)/TM (SEQ ID NO: 51))
GCCTCCCACTACTTTGAAAGACACCTGGAGTTCGAGGCCCGGACG
CTGTCCCCAGGCCACACCTGGGAGGAGGCCCCCCTGCTGACTCTC
AAGCAGAAGCAGGAATGGATCTGCCTGGAGACGCTCACCCCAGAC
ACCCAGTATGAGTTTCAGGTGCGGGTCAAGCCTCTGCAAGGCGAG
TTCACGACCTGGAGCCCCTGGAGCCAGCCCCTGGCCTTCAGGACA
AAGCCTGCAGCCCTTGGGAAGGACACC/ATTCCGTGGCTCGGCCA
CCTCCTCGTGGGCCTCAGCGGGGCTTTTGGCTTCATCATCTTAGT
GTACTTGCTGATCAACTGCAGG
In some embodiments, the hinge used in a CAR
construct of the present disclosure is a CD8a
hinge sequence set forth below, or a functional
fragment or variant thereof.
CD8a Hinge (SEQ ID NO: 33)
ACCACGACGCCAGCGCCGCGACCACCAACACCGGCGCCCACCATC
GCGTCGCAGCCCCTGTCCCTGCGCCCAGAGGCGTGCCGGCCAGCG
GCGGGGGGCGCAGTGCACACGAGGGGGCTGGACTTCGCCTGTGAT

### Natural Killer Cells

The CAR constructs of the present disclosure are specifically engineered for enhanced activity and performance in natural killer (NK) cells. The term “NK cells” can refer generally to NK cells and subtypes thereof, such as memory NK cells, memory-like (ML) NK cells, and cytokine-induced memory-like (CIML) NK cells, and variations thereof, any of which may be derived from various sources, including peripheral or cord blood cells, stem cells, induced pluripotent stem cells (iPSCs), and immortalized NK cells such as NK-92 cells.

NK Cells

NK cells are traditionally considered innate immune effector lymphocytes which mediate host defense against pathogens and antitumor immune responses by targeting and eliminating abnormal or stressed cells not by antigen recognition or prior sensitization, but through the integration of signals from activating and inhibitory receptors. NK cells are an alternative to T cells for allogeneic cellular immunotherapy since they have been administered safely without major toxicity, do not cause graft versus host disease (GvHD), naturally recognize and eliminate malignant cells, and are amendable to cellular engineering.

Memory, Memory-Like, and CIML NK Cells

In addition to their innate cytotoxic and immunostimulatory activity, NK cells constitute a heterogeneous and versatile cell subset, including persistent memory NK populations, in some cases also called memory-like or cytokine-induced-memory-like (CIML) NK cells, that mount robust recall responses. Memory NK cells can be produced by stimulation by pro-inflammatory cytokines or activating receptor pathways, either naturally or artificially (“priming”). Memory NK cells produced by cytokine activation have been used clinically in the setting of leukemia immunotherapy.

Increased CD56, Ki-67, NKG2A, and increased activating receptors NKG2D, NKp30, and NKp44 have been observed in in vivo differentiated memory NK cells. In addition, in vivo differentiation showed modest decreases in the median expression of CD16 and CD11 b. Increased frequency of TRAIL, CD69, CD62L, NKG2A, and NKp30-positive NK cells were observed in ML NK cells compared with both ACT and BL NK cells, whereas the frequencies of CD27+ and CD127+ NK cells were reduced. Finally, unlike in vitro differentiated ML NK cells, in vivo differentiated ML NK cells did not express CD25.

Cytokine-Induced Memory-Like Natural Killer Cells (CIML-NKs)

NK cells may be induced to acquire a memory-like phenotype, for example by priming (preactivation) with combinations of cytokines, such as interleukin-12 (IL-12), IL-15, and IL-18. These cytokine-induced memory-like NK cells (CIML-NK) exhibit enhanced response upon restimulation with the cytokines or engagement of activating receptors. CIML-NK cells may be produced by activation with cytokines such as IL-12, IL-15, and IL-18 and/or their related family members, or functional fragments thereof, or fusion proteins comprising functional fragments thereof.

Memory NK cells typically exhibit differential cell surface protein expression patterns when compared to conventional NK cells. Such expression patterns are known in the art and may comprise, for example, increased CD56, CD56 subset CD56dim, CD56 subset CD56bright, CD16, CD94, NKG2A, NKG2D, CD62L, CD25, NKp30, NKp44, and NKp46 (compared to control NK cells) in CIML-NK cells (see e.g., Romee et al. Sci Transl Med. 2016 Sep. 21; 8(357):357). Memory NK cells may also be identified by observed in vitro and in vivo properties, such as enhanced effector functions such as cytotoxicity, improved persistence and increased IFN-γ production when compared to a heterogenous NK cell population.

The NK cells used according to the present disclosure can be prepared using any known methodologies. For example, in some embodiments, the isolated NK cells can be activated using cytokines, such as IL-12/15/18. The NK cells can be incubated in the presence of the cytokines for an amount of time sufficient to form CIML-NK cells. For example, the amount of time sufficient to form CIML-NK cells can be between about 8 and about 24 hours, about 12 hours, or about 16 hours. As another example, the amount of time sufficient to form cytokine-activated memory-like (ML) NK cells can be at least about 1 hour; about 2 hours; about 3 hours; about 4 hours; about 5 hours; about 6 hours; about 7 hours; about 8 hours; about 9 hours; about 10 hours; about 11 hours; about 12 hours; about 13 hours; about 14 hours; about 15 hours; about 16 hours; about 17 hours; about 18 hours; about 19 hours; about 20 hours; about 21 hours; about 22 hours; about 23 hours; about 24 hours; about 25 hours; about 26 hours; about 27 hours; about 28 hours; about 29 hours; about 30 hours; about 31 hours; about 32 hours; about 33 hours; about 34 hours; about 35 hours; about 36 hours; about 37 hours; about 38 hours; about 39 hours; about 40 hours; about 41 hours; about 42 hours; about 43 hours; about 44 hours; about 45 hours; about 46 hours; about 47 hours; or about 48 hours.

In some embodiments, the chimeric antigen receptor (CAR) can then be transduced via a viral vector (e.g., lentivirus) into the CIML-NK cells in the presence of IL-15 for an amount of time sufficient to virally transduce CAR into the CIML-NK cells, resulting in CAR-transduced ML NK cells. For example, the amount of time sufficient to form CAR-transduced ML NK cells can be between about 12 hours and about 24 hours. As another example, the amount of time sufficient to virally transduce CAR into the ML NK cells (forming CAR-transduced ML NK cells) can be at least about 1 hour; about 2 hours; about 3 hours; about 4 hours; about 5 hours; about 6 hours; about 7 hours; about 8 hours; about 9 hours; about 10 hours; about 11 hours; about 12 hours; about 13 hours; about 14 hours; about 15 hours; about 16 hours; about 17 hours; about 18 hours; about 19 hours; about 20 hours; about 21 hours; about 22 hours; about 23 hours; about 24 hours; about 25 hours; about 26 hours; about 27 hours; about 28 hours; about 29 hours; about 30 hours; about 31 hours; about 32 hours; about 33 hours; about 34 hours; about 35 hours; about 36 hours; about 37 hours; about 38 hours; about 39 hours; about 40 hours; about 41 hours; about 42 hours; about 43 hours; about 44 hours; about 45 hours; about 46 hours; about 47 hours; or about 48 hours.

In some embodiments, the CAR-transduced ML NK cells can then be incubated in the presence of IL-15 for an amount of time sufficient to express the vector and to form CAR-expressing ML NK (CARML NK cells). For example, the amount of time sufficient to form CARML NK cells can be between about 3 days and about 8 days. As an example, the amount of time sufficient to form CARML NK cells can be at least about 1 day; about 2 days; about 3 days; about 4 days; about 5 days; about 6 days; about 7 days; about 8 days; about 9 days; about 10 days; about 11 days; about 12 days; about 13 days; or about 14 days.

In some embodiments, methods for preparing ML NK cells to be used according to the present disclosure include those described in WO2020/047299 and WO2020/047473, the contents of which are incorporated herein by reference in their entireties.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, subcutaneous, epidural, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating a proliferative disease, disorder, or condition, infectious disease, or immune disorder in a subject in need administration of a therapeutically effective amount of NK cell-based therapy (e.g., using genetically modified NK cells). The disclosed NK-cell based therapy can be used as a treatment for cancer (e.g., as an immunotherapy drug), for an autoimmune disease (e.g., treatment to deplete B cells), or for an infectious disease.

The scFvs described herein can be used for targeting cancer antigens associated with hematological malignancies such as AML, ALL, or Lymphoma, but can also be expanded for use in any malignancy, autoimmune, or infectious disease where a scFv can be generated against a target. For example, the constructs described herein can be used to treat or prevent autoimmunity associated with auto-antibodies (similar indications as rituximab for autoimmunity). As another example, the disclosed constructs can also be applied to virally infected cells, using a scFv that can recognize viral antigens, for example gp120 and gp41 on HIV-infected cells.

Methods described herein are generally performed on a subject in need thereof. A subject in need of the therapeutic methods described herein can be a subject having, diagnosed with, suspected of having, or at risk for developing a proliferative disease, disorder, or condition; an immune disorder; or an infectious disease. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans. For example, the subject can be a human subject.

Generally, a safe and effective amount of a NK cell-based treatment is, for example, that amount that would cause the desired therapeutic effect in a subject while minimizing undesired side effects. In various embodiments, an effective amount of a NK cell-based treatment described herein can substantially inhibit a disease, disorder, or condition, slow the progress of a disease, disorder, or condition, or limit the development of a disease, disorder, or condition.

Substantially can be any large portion up to totality. Thus “substantially blocked or inhibited”, or “substantially removed” can be nearly or nearly completely blocked, inhibited, or removed.

According to the methods described herein, administration can be parenteral, pulmonary, oral, topical, intradermal, intramuscular, intraperitoneal, intravenous, subcutaneous, intranasal, epidural, ophthalmic, buccal, or rectal administration. Preferably, NK cells can be administered as an intravenous infusion.

When used in the treatments described herein, a therapeutically effective amount of a NK cell-based treatment can be employed in a purified form or, where such forms exist, in pharmaceutically acceptable form and with or without a pharmaceutically acceptable excipient. For example, the compounds of the present disclosure can be administered, at a reasonable benefit/risk ratio applicable to any medical treatment, in a sufficient amount to inhibit a disease, disorder, or condition, slow the progress of a disease, disorder, or condition, or limit the development of a disease, disorder, or condition.

The amount of NK cell-based treatment (e.g., CARML NK cells) described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of the NK cell-based treatment can occur as a single event or over a time course of treatment. For example, NK cell-based treatment can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a disease, disorder, or condition, such as chemotherapy, immunotherapy, or checkpoint blockade therapy. For example, a subject can be administered at least one therapeutic agent selected from an interferon; a checkpoint inhibitor antibody; an antibody-drug conjugate (ADC); an anti-HLA-DR antibody; or an anti-CD74 antibody. Other examples can include a therapeutic agent selected from a second antibody or antigen-binding fragment thereof, a drug, a toxin, an enzyme, a cytotoxic agent, an anti-angiogenic agent, a pro-apoptotic agent, an antibiotic, a hormone, an immunomodulator, a cytokine, a chemokine, an antisense oligonucleotide, a small interfering RNA (siRNA), a boron compound, or a radioisotope.

A NK cell-based treatment can be administered simultaneously or sequentially with another agent, such as an antibiotic, an anti-inflammatory, or another agent. For example, a NK cell-based treatment can be administered simultaneously with another agent, such as an antibiotic or an anti-inflammatory. Simultaneous administration can occur through administration of separate compositions, each containing one or more of a NK cell-based treatment, an antibiotic, an anti-inflammatory, or another agent. Simultaneous administration can occur through administration of one composition containing two or more of a NK cell-based treatment, an antibiotic, an anti-inflammatory, or another agent. A NK cell-based treatment can be administered sequentially with an antibiotic, an anti-inflammatory, or another agent. For example, a NK cell-based treatment can be administered before or after administration of an antibiotic, an anti-inflammatory, or another agent.

Methods and compositions as described herein can be used for the prevention, treatment, or slowing the progression of cancer, autoimmune conditions associated with autoantibodies, immune disorder, or infectious diseases (e.g., bacterial, viral). The disclosed CARML NK cell constructs can be designed to incorporate a targeting antibody fragment against a disease-associated antigen, such as scFvs that target cancer or an infectious disease. As described herein, targeting antibody fragments against a disease-associated antigens are well known.

For example, the cancer can a hematological cancer or a cancer with a solid tumor. For example, the cancer can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sezary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sezary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

As another example, the autoimmune condition or immune disorder can be Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune retinopathy; Autoimmune urticaria; Axonal & neuronal neuropathy (AMAN); Belo disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressler's syndrome; Endometriosis; Eosinophilic esophagitis (EoE); Eosinophilic fasciitis; Erythema nodosum, Essential mixed cryoglobulinemia; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inverse); Hypogammalglobulinemia; IgA Nephropathy; IgG4-related sclerosing disease; Immune thrombocytopenic purpura (ITP); Inclusion body myositis (IBM); Interstitial cystitis (IC); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus; Ligneous conjunctivitis; Linear IgA disease (LAD); Lupus; Lyme disease chronic; Meniere's disease; Microscopic polyangiitis (MPA); Mixed connective tissue disease (MCTD); Mooren's ulcer; Mucha-Habermann disease; Multifocal Motor Neuropathy (MMN) or MMNCB, Multiple sclerosis; Myasthenia gravis; Myositis; Narcolepsy; Neonatal Lupus; Neuromyelitis optica; Neutropenia; Ocular cicatricial pemphigoid; Optic neuritis; Palindromic rheumatism (PR); PANDAS; Paraneoplastic cerebellar degeneration (POD); Paroxysmal nocturnal hemoglobinuria (PNH); Parry Romberg syndrome; Pars planitis (peripheral uveitis); Parsonage-Turner syndrome; Pemphigus; Peripheral neuropathy; Perivenous encephalomyelitis; Pernicious anemia (PA); POEMS syndrome; Polyarteritis nodosa; Polyglandular syndromes type I, II, Ill; Polymyalgia rheumatica; Polymyositis; Postmyocardial infarction syndrome; Postpericardiotomy syndrome; Primary biliary cirrhosis; Primary sclerosing cholangitis; Progesterone dermatitis; Psoriasis; Psoriatic arthritis; Pure red cell aplasia (PRCA); Pyoderma gangrenosurn, Raynaud's phenomenon; Reactive Arthritis; Reflex sympathetic dystrophy; Relapsing polychondritis; Restless legs syndrome (RLS); Retroperitoneal fibrosis; Rheumatic fever; Rheumatoid arthritis; Sarcoidosis; Schmidt syndrome; Scleritis; Scleroderma; Sjögren's syndrome; Sperm & testicular autoimmunity; Stiff person syndrome (SPS); Subacute bacterial endocarditis (SBE); Susac's syndrome; Sympathetic ophthalmia (SO); Takayasu's arteritis; Temporal arteritis/Giant cell arteritis; Thrombocytopenic purpura (TTP); Tolosa-Hunt syndrome (THS); Transverse myelitis; Type 1 diabetes; Ulcerative colitis (UC); Undifferentiated connective tissue disease (UCTD); Uveitis; Vasculitis; Vitiligo; or Vogt-Koyanagi-Harada Disease.

As another example the autoimmune condition or immune disorder can be an autoinflammatory disease. The autoinflammatory can be Familial Mediterranean Fever (FMF), neonatal Onset Multisystem Inflammatory Disease (NOMID), Tumor Necrosis Factor Receptor-Associated Periodic Syndrome (TRAPS), Deficiency of the Interleukin-1 Receptor Antagonist (DIRA), Behçet's Disease, or Chronic Atypical Neutrophilic Dermatosis with Lipodystrophy and Elevated Temperature (CANDLE).

As another example, the treatment of an infectious disease can be any bacterial infection or viral infection, using a scFv that can recognize antigens, such as antigens on HIV infected cells. The infectious disease can be Acute Flaccid Myelitis (AFM); Anaplasmosis; Anthrax; Babesiosis; Botulism; Brucellosis; Campylobacteriosis; Carbapenem-resistant Infection (CRE/CRPA); Chancroid; Chikungunya Virus Infection (Chikungunya); Chlamydia; Ciguatera (Harmful Algae Blooms (HABs)); Clostridium Difficile Infection; Clostridium Perfringens (Epsilon Toxin); Coccidioidomycosis fungal infection (Valley fever); Creutzfeldt-Jacob Disease, transmissible spongiform encephalopathy (CJD); Cryptosporidiosis (Crypto); Cyclosporiasis; Dengue, 1, 2, 3, 4 (Dengue Fever); Diphtheria; E. coli infection, Shiga toxin-producing (STEC); Eastern Equine Encephalitis (EEE); Ebola Hemorrhagic Fever (Ebola); Ehrlichiosis; Encephalitis, Arboviral or parainfectious; Enterovirus Infection, Non-Polio (Non-Polio Enterovirus); Enterovirus Infection, D68 (EV-D68); Giardiasis (Giardia); Glanders; Gonococcal Infection (Gonorrhea); Granuloma inguinale; Haemophilus Influenza disease, Type B (Hib or H-flu); Hantavirus Pulmonary Syndrome (HPS); Hemolytic Uremic Syndrome (HUS); Hepatitis A (Hep A); Hepatitis B (Hep B); Hepatitis C (Hep C); Hepatitis D (Hep D); Hepatitis E (Hep E); Herpes; Herpes Zoster, zoster VZV (Shingles); Histoplasmosis infection (Histoplasmosis); Human Immunodeficiency Virus/AIDS (HIV/AIDS); Human Papillomavirus (HPV); Influenza (Flu); Legionellosis (Legionnaires Disease); Leprosy (Hansens Disease); Leptospirosis; Listeriosis (Listeria); Lyme Disease; Lymphogranuloma venereum infection (LGV); Malaria; Measles; Melioidosis; Meningitis, Viral (Meningitis, viral); Meningococcal Disease, Bacterial (Meningitis, bacterial); Middle East Respiratory Syndrome Coronavirus (MERS-CoV); Mumps; Norovirus; Paralytic Shellfish Poisoning (Paralytic Shellfish Poisoning, Ciguatera); Pediculosis (Lice, Head and Body Lice); Pelvic Inflammatory Disease (PID); Pertussis (Whooping Cough); Plague; Bubonic, Septicemic, Pneumonic (Plague); Pneumococcal Disease (Pneumonia); Poliomyelitis (Polio); Powassan; Psittacosis (Parrot Fever); Pthiriasis (Crabs; Pubic Lice Infestation); Pustular Rash diseases (Small pox, monkeypox, cowpox); Q-Fever; Rabies; Ricin Poisoning; Rickettsiosis (Rocky Mountain Spotted Fever); Rubella, Including congenital (German Measles); Salmonellosis gastroenteritis (Salmonella); Scabies Infestation (Scabies); Scombroid; Septic Shock (Sepsis); Severe Acute Respiratory Syndrome (SARS); Shigellosis gastroenteritis (Shigella); Smallpox; Staphyloccal Infection, Methicillin-resistant (MRSA); Staphylococcal Food Poisoning, Enterotoxin-B Poisoning (Staph Food Poisoning); Staphylococcal Infection, Vancomycin Intermediate (VISA); Staphylococcal Infection, Vancomycin Resistant (VRSA); Streptococcal Disease, Group A (invasive) (Strep A (invasive)); Streptococcal Disease, Group B (Strep-B); Streptococcal Toxic-Shock Syndrome, STSS, Toxic Shock (STSS, TSS); Syphilis, primary, secondary, early latent, late latent, congenital; Tetanus Infection, tetani (Lock Jaw); Trichomoniasis (Trichomonas infection); Trichonosis Infection (Trichinosis); Tuberculosis (TB); Tuberculosis (Latent) (LTBI); Tularemia (Rabbit fever); Typhoid Fever, Group ID, Typhus; Vaginosis, bacterial (Yeast Infection); Vaping-Associated Lung Injury (e-Cigarette Associated Lung Injury); Varicella (Chickenpox); Vibrio cholerae (Cholera); Vibriosis (Vibrio); Viral Hemorrhagic Fever (Ebola, Lassa, Marburg); West Nile Virus; Yellow Fever; Yersenia (Yersinia); or Zika Virus Infection (Zika).

Administration

An aspect of the present disclosure provides for NK cells (e.g., CARML NK cells, modified NK cells, pre-activated NK cells, NKG2A-blocked NK cells, pre-activated and NKG2A-blocked NK cells) to be directly administered to a subject.

Apheresis (e.g., the removal of blood plasma from the body by the withdrawal of blood, its separation into plasma and cells, and the reintroduction of the cells) can be performed on the subject.

In some embodiments, the NK cells can be purified and activated with IL-12/IL-15/IL-18 for about 12 hours. The NK cells can be washed and transduced with CAR lentivirus (e.g., twice over about two days). The cells can be washed and infused into the patient at about 10⁷ cell/kg. In the haplo/allo setting the cells can be supported with rhIL-2 and in the autologous setting the cells can be supported with IL-15.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

The following descriptions are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription. (Specify somewhere the promoters of most interest)

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by and/or supports the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell. In another example, the domains present in a CAR construct according to the present disclosure can be said to be operably linked to one another so long as they carry out their intended function within the CAR.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked to another.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### Examples

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Improved Intracellular Signaling Domains for Use in Chimeric Antigen Receptor Memory-Like (CARML) NK Cells

Immunological cellular therapies represent a revolutionary step in how cancer and other conditions can be treated. A newly emerging area of promise centers on the use of natural killer (NK) cells for cellular therapies due to their improved safety profile over existing CAR-T products, as well as their ability to be used as an allogeneic “off-the-shelf” product without genetic manipulation. However, there are some challenges to be overcome. For example, a higher dose of NK cells is typically required to achieve comparable levels of tumor clearance compared with CAR-T cells. Additionally, there are certain cancers that show resistance to killing by NK cell therapies due to the lack of canonical NK activating ligands (ULBP1, MIC-A/B, etc), via the expression of immunosuppressive mediators (PGE2, TGFb, etc), and/or by the expression of proteins which bind inhibitory NK receptors (HLA-E). Furthermore, due to the heterogeneous nature of PBMC-derived NK cells, some donor-to-donor variability has been observed when testing NK cytotoxic potential against cancer cells.

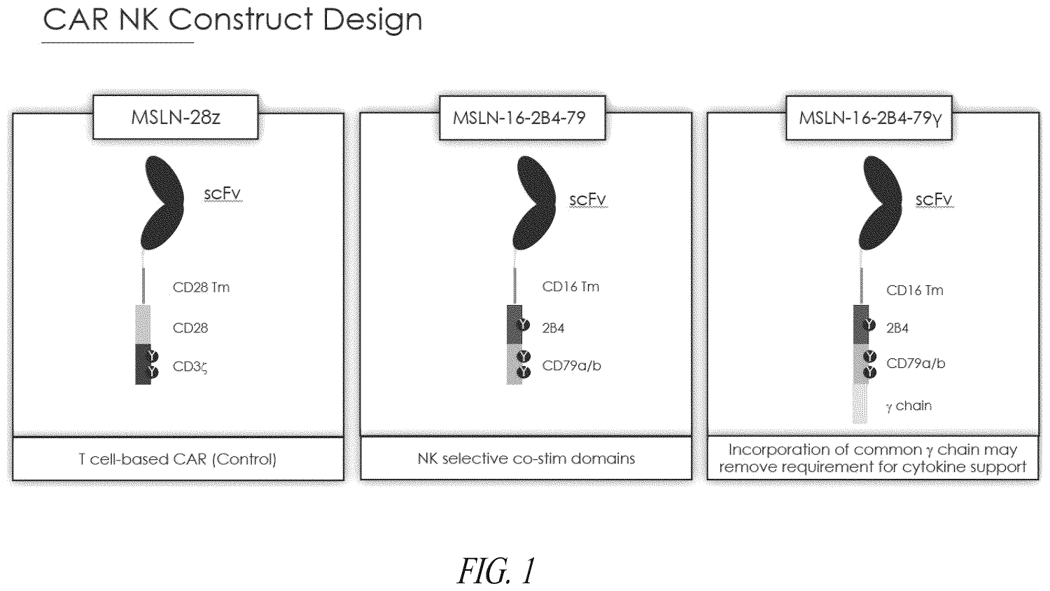

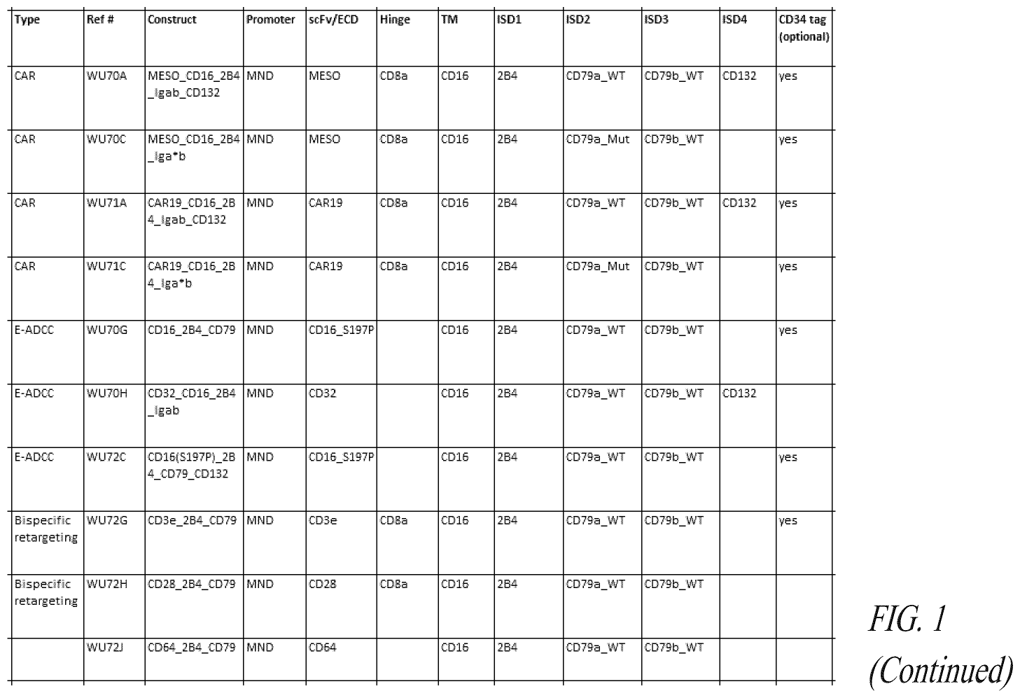

We investigated a panel of various intracellular signaling and other CAR domains in NK cells to determine their effects on solid tumor cell killing. Based on these studies, we identified unique combinations of intracellular signaling domains having superior activity to the rest. A number of illustrative CAR constructs prepared according to the present disclosure can be seen in FIG. 1.

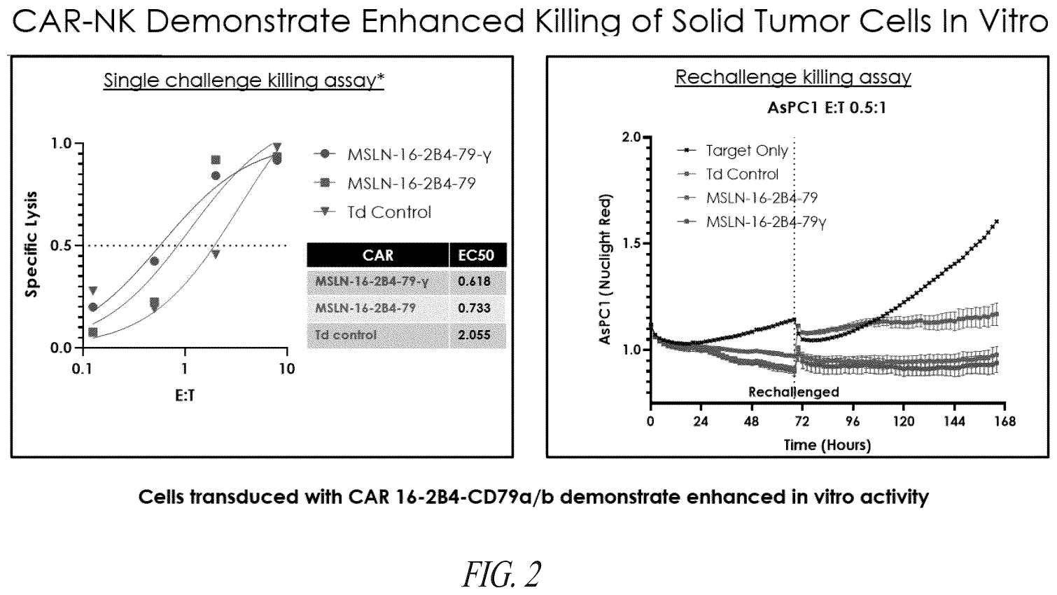

We have found that CAR constructs containing intracellular signaling domains from CD79A, CD79B and/or 2B4 gave rise to enhanced killing of the pancreatic tumor cell line Aspc1 as well as the mesothelioma cell line H226 (FIG. 2). CARs containing these intracellular domains advantageously bypass some of the problems associated conventional NK cells, at least in part by providing a more potent chimeric receptor compared to the endogenous receptors NK cells typically encode. This results in a genetically modified NK cell product that is 1) more effective at lower doses, 2) more resilient to tumor immuno-suppression and evasion, and 3) has a better defined and uniform cytotoxicity profile.

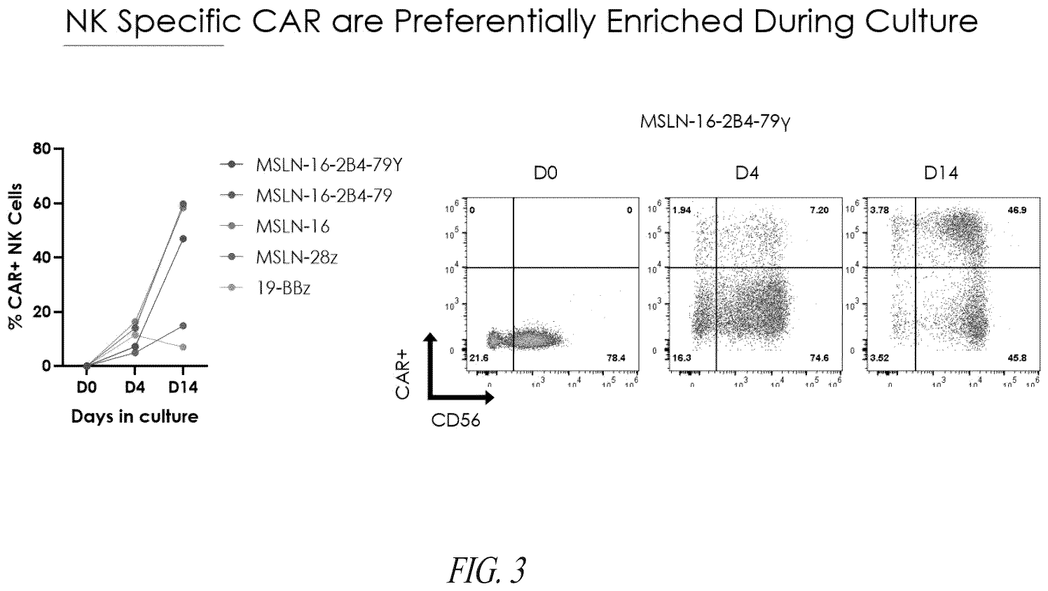

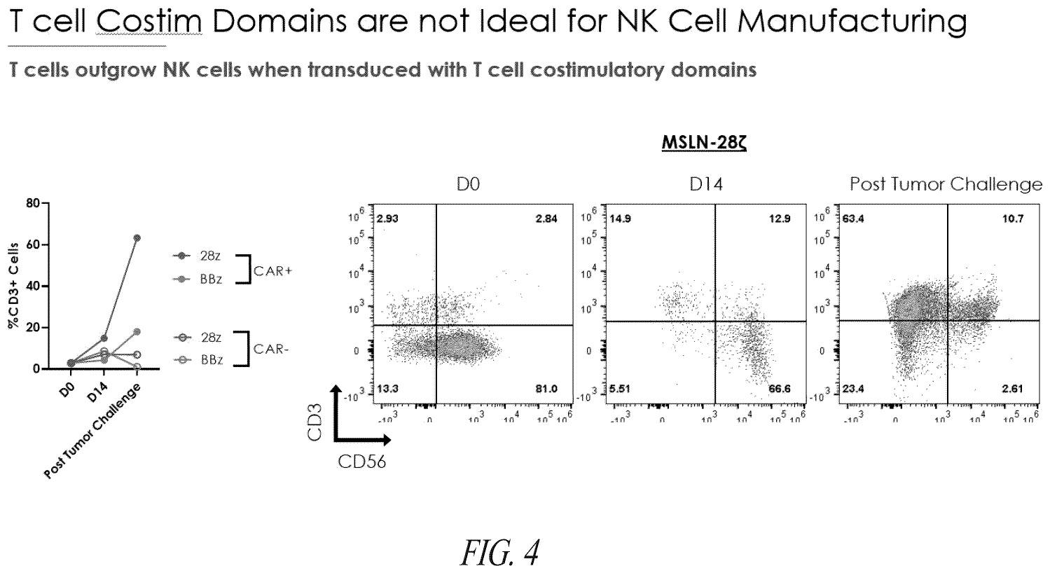

Also, NK cells that were transduced with these intracellular signaling domains demonstrated an unexpected ability to self-enrich during manufacturing (FIG. 3). Additionally, we found that transduction of NK cell cultures with a CD28-CD3z CAR resulted in expansion of basal contaminant T cell populations during manufacturing, demonstrating that NK cell-specific intracellular signaling domains are superior for the production of highly enriched, PBMC-derived allogeneic NK cell immunotherapeutics (FIG. 4).

### Example 2: Antibody-Dependent Cellular Cytotoxicity (ADCC)-Enabled CARS for Use in Chimeric Antigen Receptor Memory-Like (CARML) NK Cells

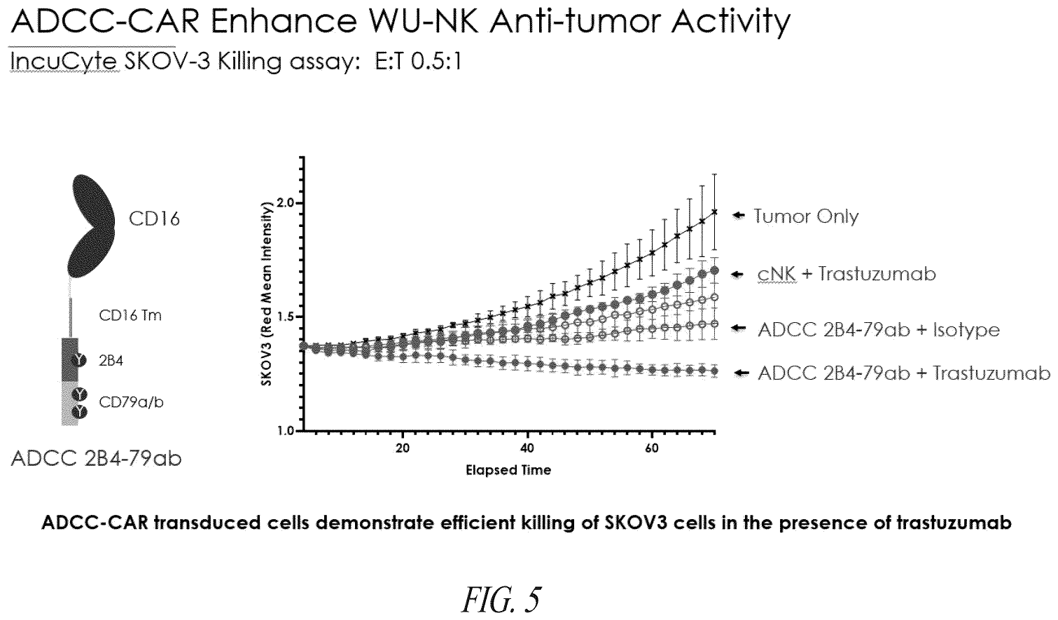

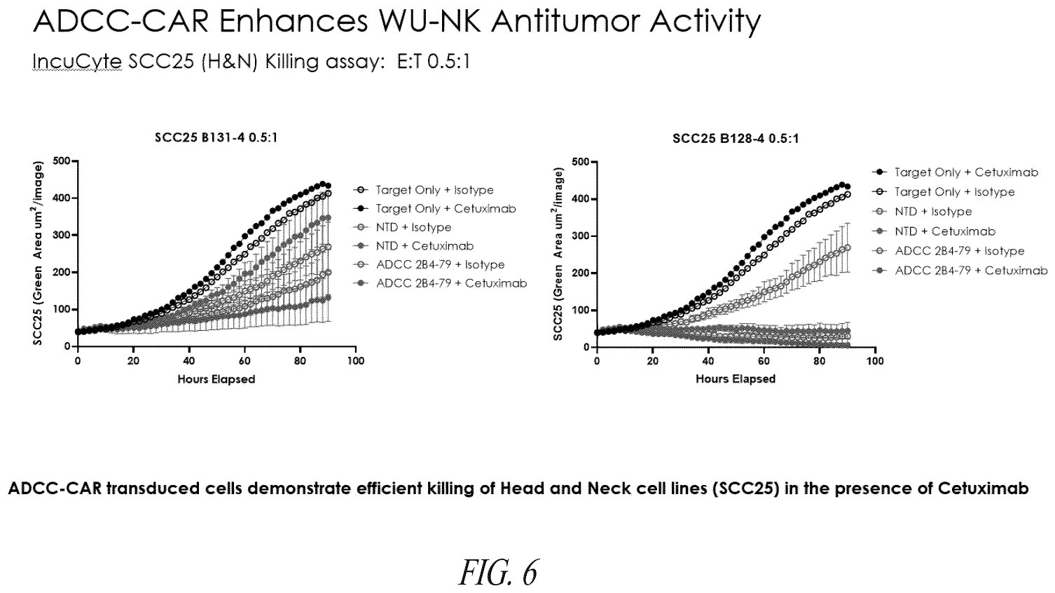

This example describes ADCC-enabled NK cells containing chimeric receptor constructs with optimized intracellular signaling domains. ADCC depends on the binding of the cell surface receptor (CD16) on NK cells to an antibody that recognizes a tumor antigen. ADCC can be inhibited by cleavage of the extracellular domain of CD16 from the cell surface by the ADAM17 protease. We designed a transgene to enhance ADCC that incorporates a mutation at the ADAM17 cleavage site to prevent CD16 cleavage (S197P). In addition to the enhanced CD16 extracellular domain, our ADCC-CAR (E-ADCC) utilizes the same intracellular costimulatory domains (2B4, CD79A, CD79B) as above. The E-ADCC NK cells exhibited increased cytotoxicity towards SKOV3 cancer cells in conjunction with Trastuzumab (FIG. 5) and toward SCC26 cancer cells in conjunction with Cetuximab (FIG. 6) compared to control NK cells. Additionally, similarly to the other CAR constructs employing these intracellular signaling domains in NK cells, we observed self-enrichment of E-ADCC NK cells during our manufacturing process.

The use of extracellular domains derived from molecules such as CD16, CD3e and CD28 offers a high degree of flexibility to the use of chimeric receptors. Traditionally, CARs have been designed with a single antigen-targeting scFv extracellular domain. As a result, in order to target a different tumor antigen, it was necessary to design, develop and manufacture a new CAR construct. In contrast, the use of extracellular domains from cellular receptors such as CD16, CD3E and CD28 allows for the use of combination therapies in conjunction with an E-ADCC enabled NK cell. For example, a combination of a therapeutic antibody with a CD16 based E-ADCC NK is envisioned. In another example, a combination of a BiTE with a CD3e and/or CD28 based E-ADCC NK cell is expected to enhance targeted cytotoxicity. A more modular approach to targeting cancers such as this can reduce the need to make different cellular drug products for different cancers.

### Example 3: CD3 Chimeric Receptor Expressing NK Cells Enable New Strategies to Target Different Cancer with Reduced Toxicity

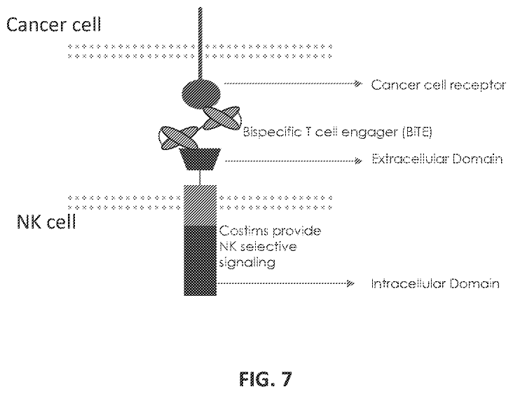

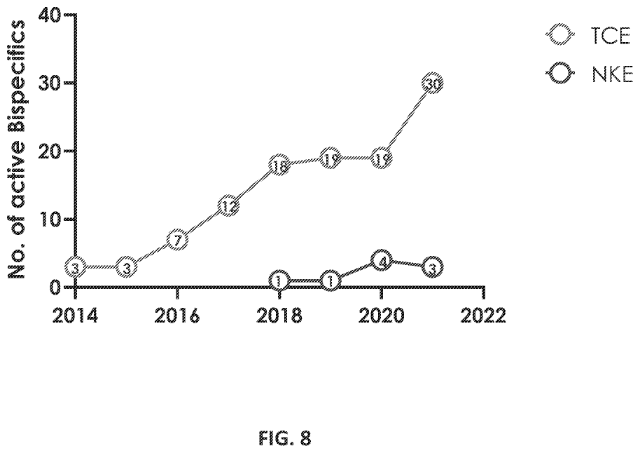

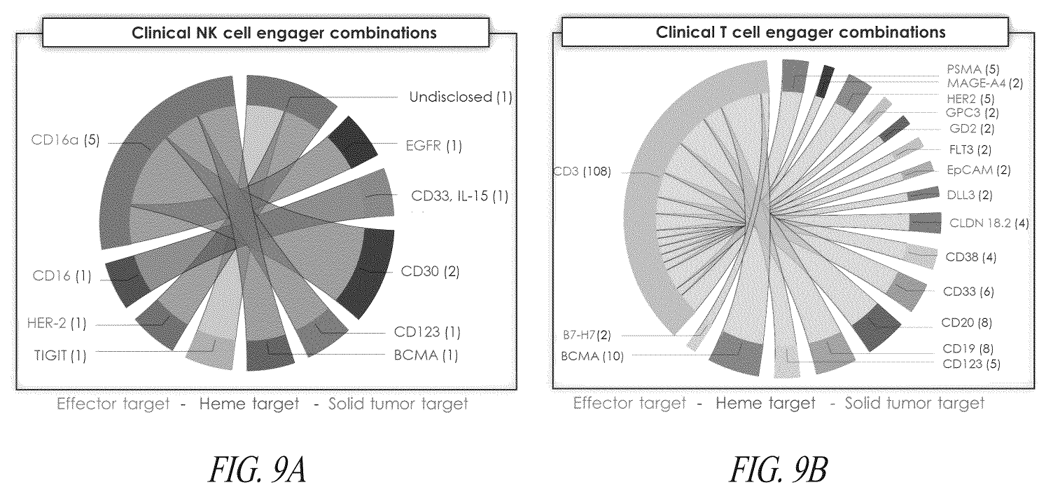

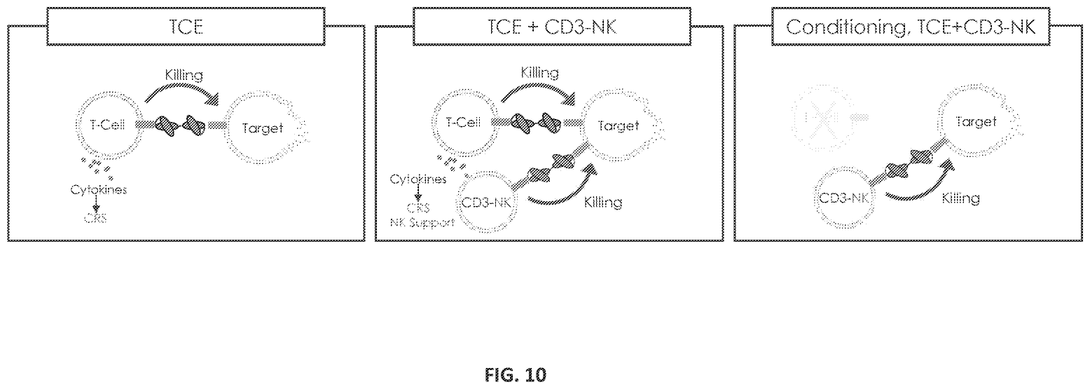

FIG. 7 shows different strategies for using NK cells to target cancer. CD3 chimeric receptor expressing NK cells (CD3-NK cells) would enable use of bispecific T cell engagers (BiTEs) to direct CD3-NK cell killing of cancer cells. The combination of CD3-NK cells would allow for use of NK cells in novel indications with target flexibility. Additional, use of a CD3 chimeric receptor with optimized intracellular domains would provide signaling selective to NK cells, improving function. While there are NK cell engagers in development, FIG. 8, FIG. 9 and FIG. 10 illustrate that T cell engagers (TCEs), such as BiTEs, dominate the retargeting landscape. FIG. 8 shows the number of bispecific TCEs far outnumber that of NK cell engagers (NKEs). Additionally, FIG. 9A shows that NKEs are not only few in numbers but limited in tumor target antigens while FIG. 9B shows that TCEs are plentiful and cover a diverse tumor antigen target repertoire. Moreover, as shown in FIG. 10, TCEs activate endogenous T cells and pose a high risk of cytokine release syndrome (CRS) and might not overcome the suppressive tumor microenvironment (TME). CD3-NK cells, used in combination with TCEs, would enable a coordinated antitumor attack and increase anti-tumor activity and help in overcoming the suppressive TME. Alternatively, a conditioning regimen to eliminate endogenous T cells would minimize risk of CRS while engineered CD3-NK cells used in combination with TCEs can still effectively kill tumor cells.

### Example 4: Stable Cell Surface Expression of CD3e Containing Receptors

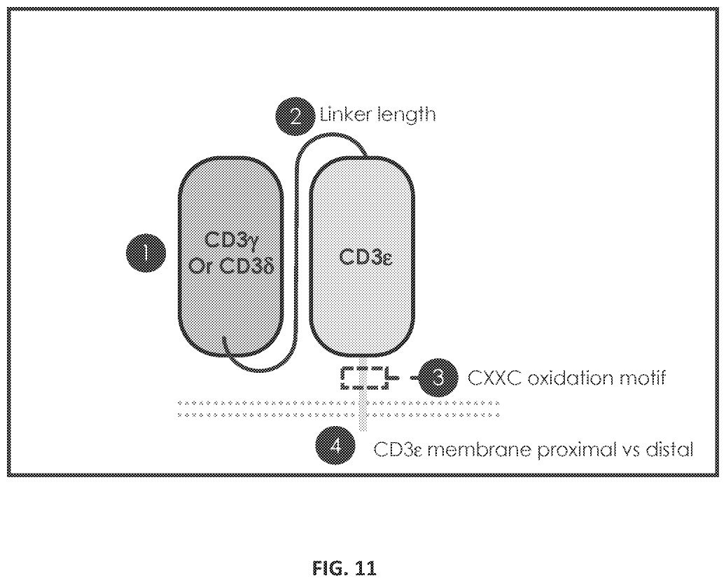

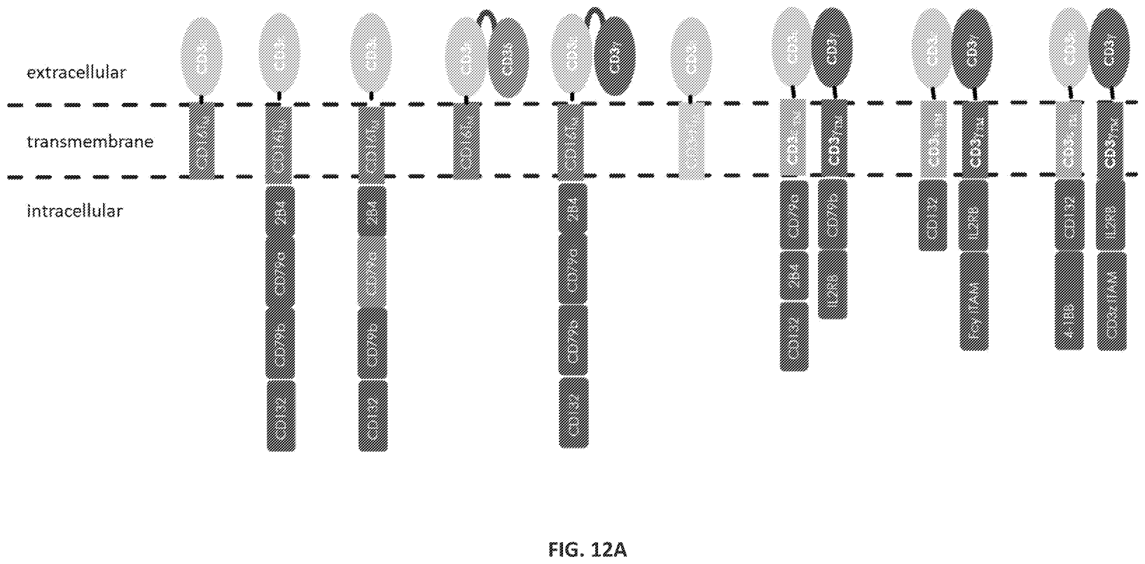

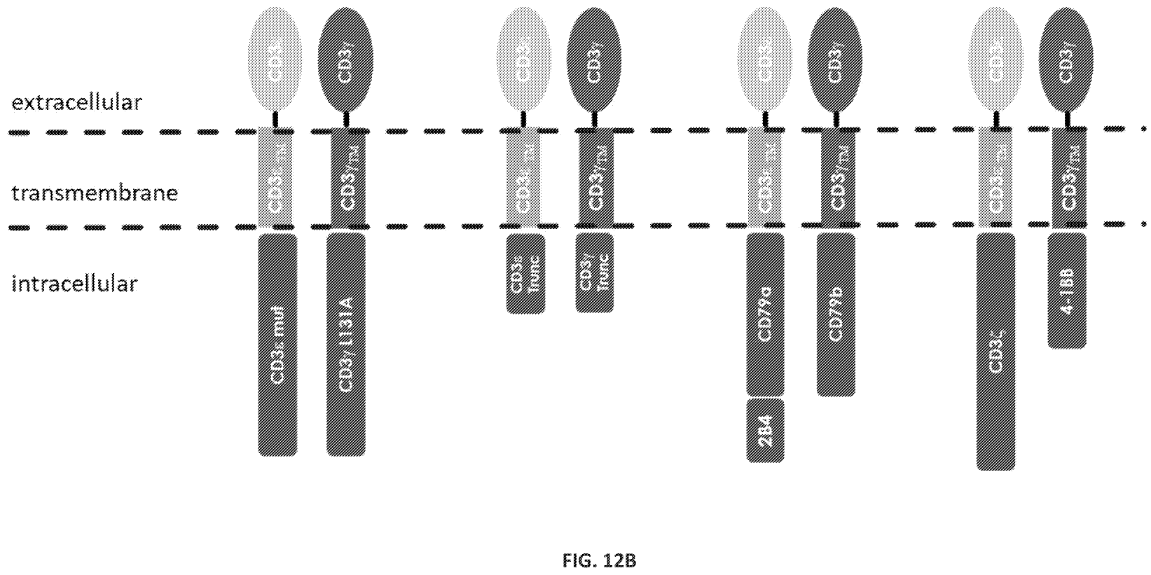

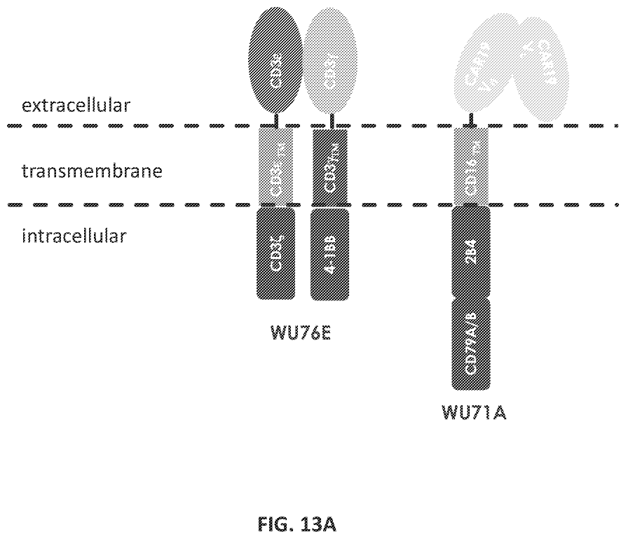

Since most BiTEs target the CD3e chain of the CD3-TCR complex but CD3e cannot traffic to the NK cell surface on its own, FIG. 11 shows a strategy for stabilizing cell surface expression of CD3e as a single chain variable fragment (Fv) by co-expression with other CD3 members through a linker and through altering the highly conserved CXXC motif found on the membrane proximal end of CD3e, CD3g or CD3d and through varying the position of CD3e as the membrane proximal or distal domain within the extracellular region. Utilizing the aforementioned strategies, an exemplary bicistronic chimeric receptor construct for expression of CD3e on the cell surface is shown below and several illustrative chimeric receptor and bicistronic chimeric receptor constructs for expression of CD3e on the cell surface are shown in FIG. 12A and FIG. 12B.

Bicistronic receptor_WU76E
(SEQ ID NO: 52)
MQSGTHWRVLGLCLLSVGVWGQDGNEEMGGITQTPYKVSIS
GTTVILTCPQYPGSEILWQHNDKNIGGDEDDKNIGSDEDH
LSLKEFSELEQSGYYVCYPRGSKPEDANFYLYLRARVCEN
CMEMDVMSVATIVIVDICITGGLLLLVYYWSRVKFSRSAD
APAYKQGQNQLYNELNLGRREEYDVLDKRRGRDPEMGGKP
RRKNPQEGLYNELQKDKMAEAYSEIGMKGERRRGKGHDGL
YQGLSTATKDTYDALHMQALPPRGSGATNFSLLKQAGDVE
ENPGPMEQGKGLAVLILAIILLQGTLAQSIKGNHLVKVYD
YQEDGSVLLTCDAEAKNITWFKDGKMIGFLTEDKKKWNLG
SNAKDPRGMYQCKGSQNKSKPLQVYYRMCQNCIELNAATI
SGFLFAEIVSIFVLAVGVYFIAKRGRKKLLYIFKQPFMRP
VQTTQEEDGCSCRFPEEEEGGCEL

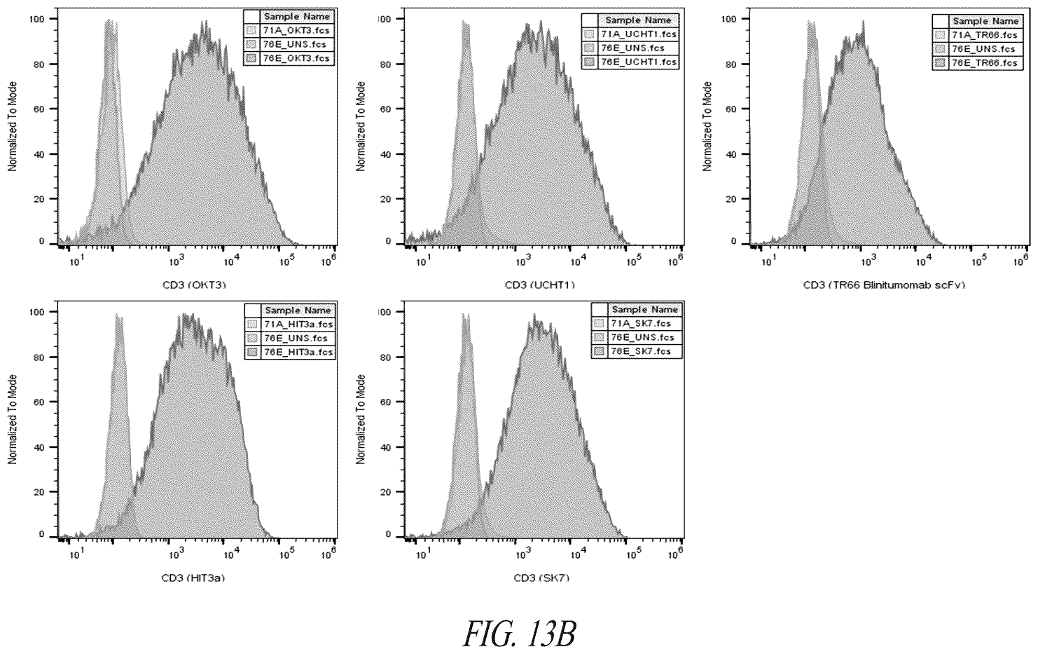

To generate CD3-NK cells, NK cells were isolated using STEMCELL kits and primed on day 0. Exogenous factors were then added to further expand the NK cell culture on day 1. On day 3, NK cells were transduced with lentivirus plasmid constructs expressing the desired chimeric receptor through an 8 hour co-culture in a cell culture vessel. Given that CD3e cannot traffic to the cell membrane on its own, experiments were performed to assess the feasibility of expressing CD3e on the cell surface. 293T-X cells were transfected with lentivirus plasmid constructs expressing the chimeric receptors WU76E or WU71A shown in FIG. 13A. Forty-eight hours post transfection, 293T-X cells were stained using 1 of 5 anti-CD3 antibody clones that target CD3e: OKT3, UCHT1, TR66, HIT3a, or SK7 and assessed for cell surface expression of CD3 by flow cytometry. As shown in FIG. 13B, CD3e expression was detected on 293T-X cells expressing the WU76E construct comprising a CD3e extracellular domain but not on the cells expressing the negative control WU71A construct comprising a CD19 extracellular domain.

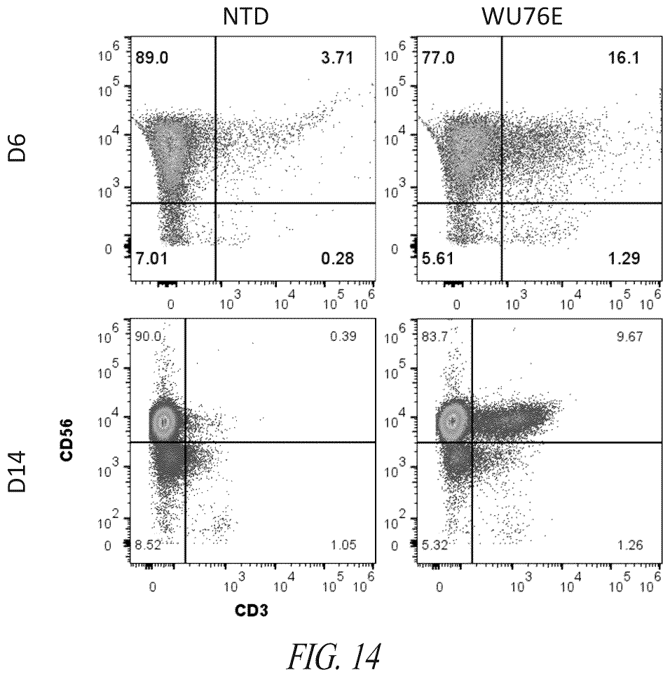

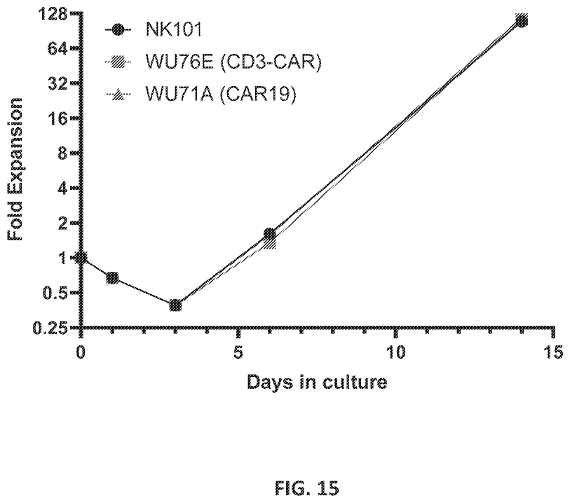

To assess expression of CD3e on human NK cells, non-transduced NK cells (NTD), or NK cells virally transduced with the WU76E CD3e expressing construct (WU76E-NK cells) were stained with anti-CD3e and anti-CD56 antibodies on day 6 or day 14 of manufacturing. Percent of cells with cell surface expression of CD3e and CD56 was quantified by flow cytometry. FIG. 14 shows stable CD3e expression on the cell surface of NK cells transduced with WU76E and FIG. 15 shows that NK cells transduced with WU76E expanded in culture similarly to non-transduced NK cells (NK101) and NK cells transduced with WU71A (WU71A-NK cells). Taken together, Example 3 demonstrates that CD3e can traffic and be expressed on the cell surface of NK cells in the absence of the complete TCR complex. Furthermore, the expression of CD3 on the cell surface did not inhibit NK cell growth.

### Example 5: NK Cells Expressing CD3e Chimeric Receptors Display Potent Cytotoxicity Against Tumor Cells in Combination with a BiTE

Experiments were performed to assess the ability of WU76E NK cells to kill tumor cells in the presence of Blinatumomab, a BiTE which targets CD19 and CD3.

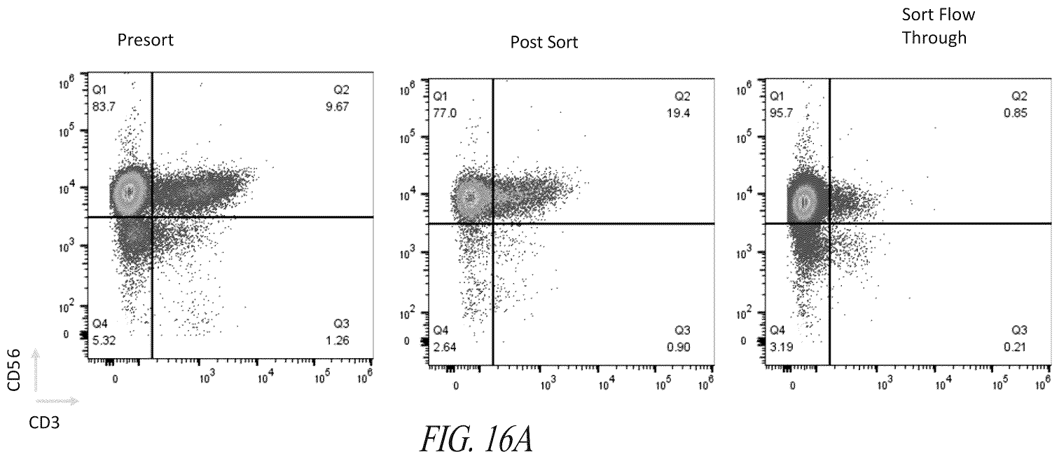

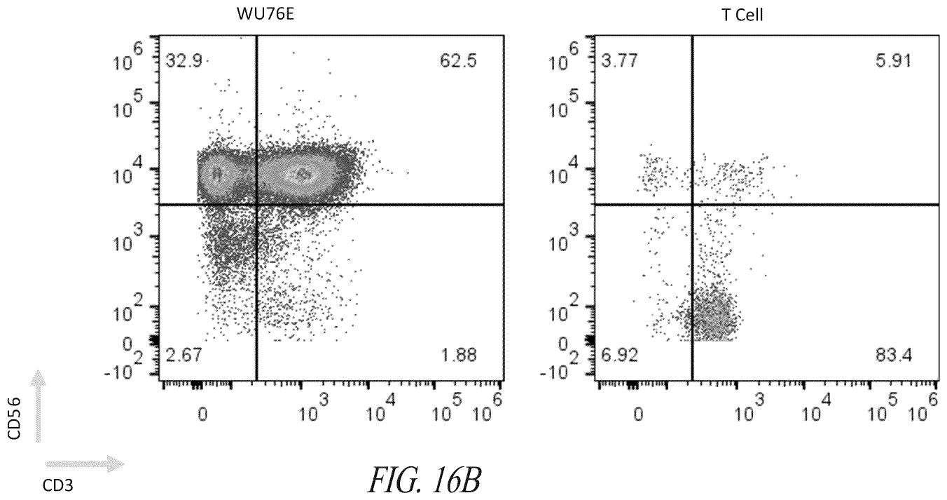

STEMCELL Human CD3 Positive Selection kits were used to positively select for CD3+ WU76E-NK cells on day 13 of manufacturing. Cell surface expression of CD56 and CD3 on WU76E-NK cells were quantified by flow cytometry on three populations: WU76E-NK cells prior to CD3 positive selection (Presort), WU76E-NK cells that were positively selected (Post Sort) and the flow through population that was not selected. As shown in FIG. 16A, purity of CD3+ WU76E-NK cells immediately post cell enrichment appeared low at 19.4% due to epitope blocking by the CD3 selection antibody cocktail, which dampened the binding of the anti-CD3 antibody used for flow cytometry staining. However, using T cells as a negative staining control, FIG. 16B shows CD3+ WU76E-NK cells were indeed enriched after the effects of epitope blocking weaned 24 hours after positive selection.

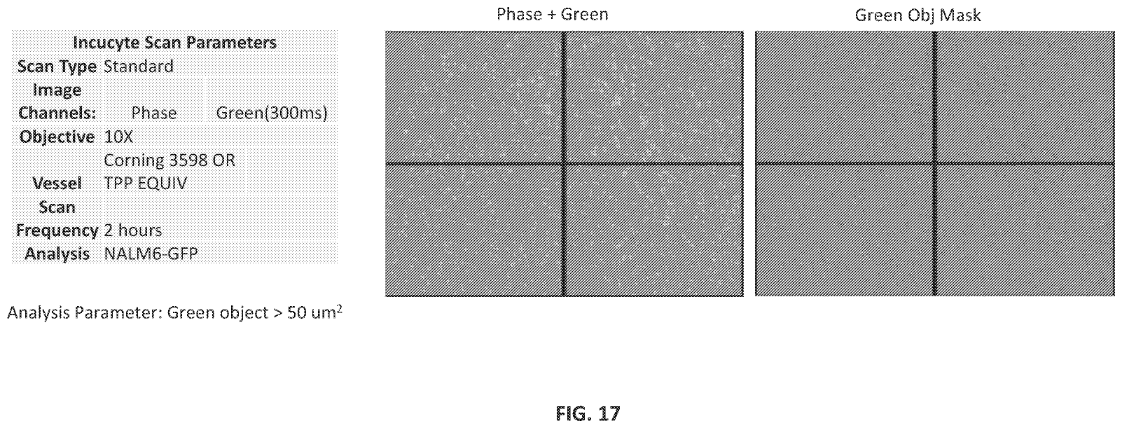

To test the cytotoxicity of CD3+ WU76E NK cells in combination with Blinatumomab, GFP+ NALM6 cancer cells expressing CD19 were selected as targets for killing. Cells were cultured and imaged with an Incucyte using the 10X objective every two hours. Green objects greater than 50 μm² in size were identified as NALM6-GFP cells and quantified. FIG. 17 shows the actual phase and green signal (GFP) images captured and the green object mask analysis done by the Incucyte software to discern viable NALM6 cells.

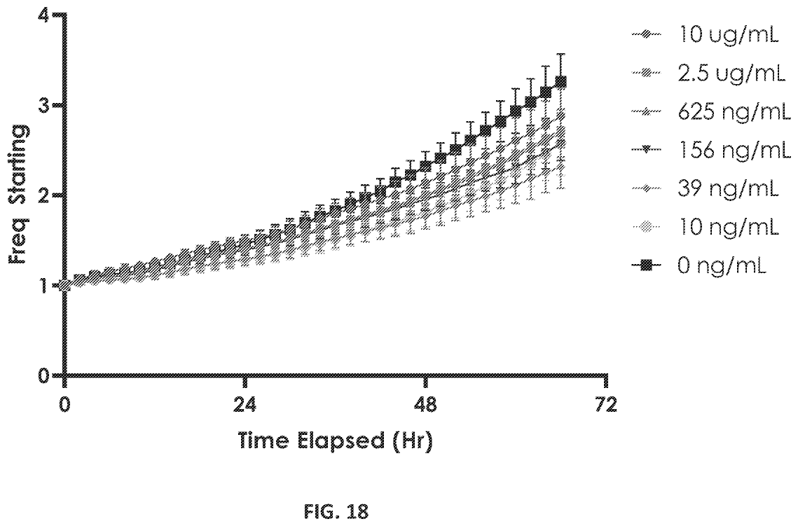

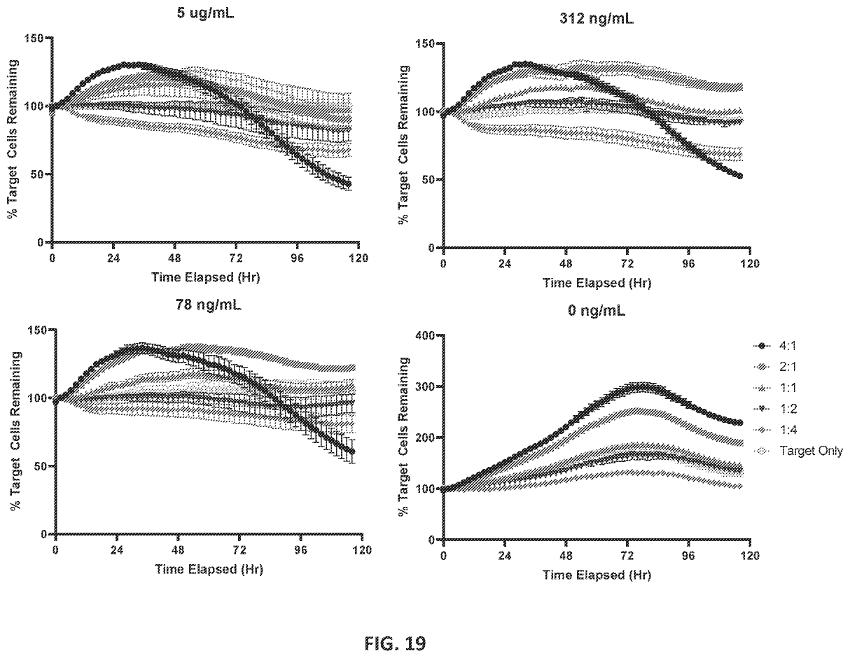

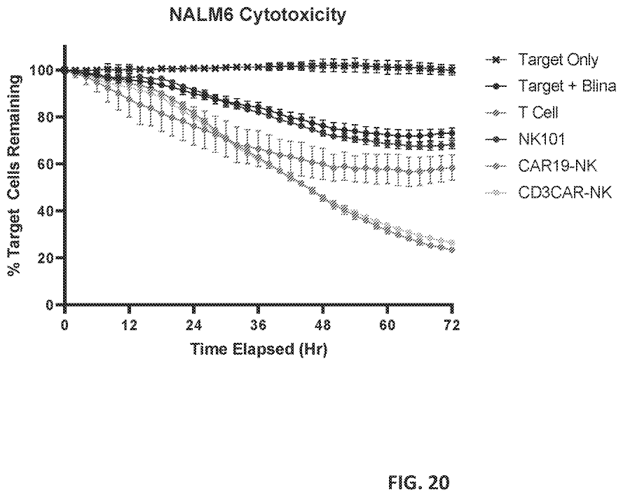

NALM6 cells were cultured in the presence of different concentrations of Blinatumomab in complete RPMI media to determine the effects of Blinatumomab alone on cell growth and determine a suitable concentration for use with WU76E-NK cells. Cell growth was tracked via Incucyte and normalized to the number of starting cells (2×10⁵ cells/well). As shown in FIG. 18, there were small decreases in cell growth across the tested blinatumomab concentrations but the NALM6 cells still expanded. NALM6 cells were then co-cultured with different ratios of naïve T cells in the presence of 5 μg/mL, 312 ng/mL, 78 ng/mL or Ong/mL of Blinatumomab and growth of NALM6 cells were tracked via Incucyte and normalized to the number of starting cells (2×10⁵ cells/well). FIG. 19 shows that while naïve T cells alone cannot kill NALM6 cells, they do kill NALM6 cells in the presence of Blinatumomab, most evident by the drop in percent of target cells remaining after 72 hours of co-culturing at the 4:1 effector to target ratio across all concentrations of Blinatumomab.

Lastly, isolated NK cells that were primed, expanded, and transduced with lentivirus expressing WU76E as described in example 4 and subsequently positively selected for CD3 on day 14 of culturing as described earlier in this example were used on day 15 for cytotoxicity assays. Briefly, 2×10⁴ NALM6 cells per well were cultured in six different conditions:
(1) target only—NALM6 cells alone,
(2) target+Blina, NALM6 cells with 100 ng/mL of Blinatumomab,
(3) T Cell—NALM6 cells co-cultured with naïve, isolated CD3+ T cells at a 1:3 effector to target ratio in the presence of 100 ng/mL of Blinatumomab,
(4) NK101—NALM6 cells co-cultured with NK101 at a 1:3 effector to target ratio in the presence of 100 ng/mL of Blinatumomab,
(5) CAR19-NK—NALM6 cells co-cultured with WU71A-NK cells at a 1:3 effector to target ratio in the presence of 100 ng/mL of Blinatumomab, and
(6) CD3CAR-NK—NALM6 cells co-cultured with WU76E-NK cells at a 1:3 effector to target ratio in the presence of 100 ng/mL of Blinatumomab.

All T and NK cells were isolated from the same donor. As shown in FIG. 20, WU76E-NK cells were effective at killing NALM6 cells in the presence of Blinatumomab and their cytotoxicity activity was comparable to that of WU71A-NK cells expressing a chimeric receptor directly targeting CD19.

1.-30. (canceled)

31. A chimeric antigen receptor (CAR) capable of being expressed in a natural killer (NK) cell, where the CAR comprises at least two of a CD79A intracellular signaling domain, a CD79B intracellular signaling domain, a 2B4 intracellular signaling domain and a DAP10 intracellular signaling domain.

32. The CAR of claim 31, where the intracellular signaling domain comprises
I) a CD79A sequence encoded by SEQ ID NO: 1 (WT), a sequence encoded by SEQ ID NO: 2 (CD79A (S197A, 5203A, T209V), or a functional fragment or variant thereof having at least 90% identity to a sequence encoded by SEQ ID NO: 1 or SEQ ID NO: 2;
II) a CD79B sequence encoded by SEQ ID NO: 3, or a functional fragment or variant thereof having thereof at least 90% identity to a sequence encoded by SEQ ID NO: 3;
III) a 2B4 sequence encoded by SEQ ID NO: 4, or a functional fragment or variant thereof having at least 90% identity to a sequence encoded by SEQ ID NO: 4; or
IV) a DAP10 sequence encoded by SEQ ID NO: 5, or a functional fragment or variant thereof having at least 90% identity to a sequence encoded by SEQ ID NO: 5.

33. The CAR of claim 31, where the CAR comprises
i) a CD79A intracellular signaling domain encoded by SEQ ID NO: 1 or SEQ ID NO: 2, a CD79B intracellular signaling domain encoded by SEQ ID NO: 3 and a 2B4 intracellular signaling domain encoded by SEQ ID NO: 4; or
ii) a CD79A intracellular signaling domain encoded by SEQ ID NO: 1 or SEQ ID NO: 2, a CD79B intracellular signaling domain encoded by SEQ ID NO: 3 and a DAP10 intracellular signaling domain encoded by SEQ ID NO: 5.

34. The CAR of claim 31, where the CAR comprises an extracellular domain capable of binding a target polypeptide.

35. The CAR of claim 34, where extracellular domain comprises an scFv sequence capable of binding the target polypeptide.

36. The CAR of claim 35, where the target polypeptide is selected from the group consisting of CD2, CD5, CD7, MSLN, CEA, PSMA, CD19, CD28, CD3, CD33, CD38, CD138, CLL-1, C-KIT CD123, CD133, CD20, BCMA, EGFR, CD3, CD4, BAFF-R, EGFR, HER2, GD2 gp120 and gp41.

37. The CAR of claim 34, where the extracellular domain comprises an Fc receptor sequence.

38. The CAR of claim 37, where the Fc receptor sequence is a CD16, CD32 or CD64 receptor sequence.

39. The CAR of claim 38, where the CD16 Fc receptor sequence has a S197P mutation.

40. The CAR of claim 34, where the extracellular domain comprises CD3e or CD28.

41. The CAR of claim 31, where the CAR comprises a transmembrane domain selected from the group consisting of CD28, CD16, CD32, CD64, NKG2D, FcγRIIIa, NKp44, NKp30, NKp46, actKIR, NKG2C, CD8a and IL15.

42. The CAR of claim 41, where the transmembrane domain comprises a CD16 transmembrane domain.

43. The CAR of claim 31, further comprising an intracellular signaling domain selected from the group consisting of CD132, CD137/41BB, DNAM-1, NKp80, 2B4, NTBA, CRACC, CD2, CD27, integrins, IL-15R, IL-18R, IL-12R, IL-21R and IRE1a.

44. The CAR of claim 31, where the CAR comprises a hinge domain selected from the group consisting of a CD8a hinge domain, a NKG2 hinge domain or a TMα hinge domain.

45. The CAR of claim 31, where the CAR is expressed under the control of a promoter that is transcriptionally active in NK cells.

46. The CAR of claim 45, where the promoter is MND.

47. The CAR of claim 31, further comprising a P2A truncated CD34 protein on the terminal end of the chimeric receptor.

48. A vector comprising a nucleic acid encoding a CAR of claim 31.

49. The vector of claim 48, where the vector is a viral vector.

50. The vector of claim 49 where the viral vector is a retroviral or lentiviral vector.

51. A genetically modified NK cell comprising (i) a CAR of claim 31, or (ii) a vector comprising a nucleic acid encoding a CAR of claim 31.

52. The genetically modified NK cell of claim 51, where the cell is deficient for NKG2A and/or CD8 expression, activity or signaling.

53. The genetically modified NK cell of claim 51, where the NK cell is derived from cord blood, peripheral blood, an immortalized cell line or an iPSC.

54. The genetically modified NK cell of claim 51, where the NK cell is memory-like NK cell.

55. A method of inducing an immune response to a disease in a subject in need thereof comprising administering to the subject (i) a CAR of claim 31, (ii) a vector comprising a nucleic acid encoding a CAR of claim 31 or (iii) a genetically modified cell comprising a CAR of claim 31 or a vector comprising a nucleic acid encoding a CAR of claim 31.

56. The method of claim 55, where the disease is cancer, an autoimmune condition or an infectious disease.


SEQUENCE LISTING (52 sequences)

SEQ ID NO: 1 - DNA, 183 bp, synthetic construct
  source 1..183: mol_type = other DNA
cgcaagcgctggcagaacgagaagctgggcctggacgccggcgacgagtacgaggacgagaacctgtacgagggcctgaacctggacgactgctccatgtacgaggacatctcccgcggcctgcagggcacctaccaggacgtgggctccctgaacatcggcgacgtgcagctggagaagccc

SEQ ID NO: 2 - DNA, 183 bp, synthetic construct
  source 1..183: mol_type = other DNA
cgcaagcgctggcagaacgagaagctgggcctggacgccggcgacgagtacgaggacgagaacctgtacgagggcctgaacctggacgactgcgccatgtacgaggacatcgcccgcggcctgcagggcgtgtaccaggacgtgggctccctgaacatcggcgacgtgcagctggagaagccc

SEQ ID NO: 3 - DNA, 147 bp, synthetic construct
  source 1..147: mol_type = other DNA
ctggacaaggacgactccaaggccggcatggaggaggaccacacctacgagggcctggacatcgaccagaccgccacctacgaggacatcgtgaccctgcgcaccggcgaggtgaagtggtccgtgggcgagcaccccggccaggag

SEQ ID NO: 4 - DNA, 360 bp, synthetic construct
  source 1..360: mol_type = other DNA
tggcgccgcaagcgcaaggagaagcagtccgagacctcccccaaggagttcctgaccatctacgaggacgtgaaggacctgaagacccgccgcaaccacgagcaggagcagaccttccccggcggcggctccaccatctactccatgatccagtcccagtcctccgcccccacctcccaggagcccgcctacaccctgtactccctgatccagccctcccgcaagtccggctcccgcaagcgcaaccactccccctccttcaactccaccatctacgaggtgatcggcaagtcccagcccaaggcccagaaccccgcccgcctgtcccgcaaggagctggagaacttcgacgtgtactcc

SEQ ID NO: 5 - DNA, 72 bp, synthetic construct
  source 1..72: mol_type = other DNA
ctgtgcgcccgcccccgccgctcccccgcccaggaggacggcaaggtgtacatcaacatgcccggccgcggc

SEQ ID NO: 6 - DNA, 267 bp, Homo sapiens
  source 1..267: mol_type = genomic DNA
ttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacc

SEQ ID NO: 7 - DNA, 126 bp, Homo sapiens
  source 1..126: mol_type = genomic DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 8 - DNA, 182 bp, Homo sapiens
  source 1..182: mol_type = genomic DNA
aaggagaaggagagagagaagagatctatttacagagtcctgggatacacagaaggcacccaataactatagaagtcccatctctaccagtcaacctaccaatcaatccatggatgatacaagagaggatatttatgtcaactatccaaccttctctcgcagaccaaagactagagtttaag

SEQ ID NO: 9 - DNA, 512 bp, Homo sapiens
  source 1..512: mol_type = genomic DNA
tttctcagggagtattgctaaaatgccaaaaaggaagttgttcaaatgccactcagtatgaggacactggagatctaaaagtgaataatggcacaagaagaaatataagtaataaggacctttgtgcttcgagatctgcagaccagacagtactatgccaatcagaatggctcaaataccaagggaagtgttattggttctctaatgagatgaaaagctggagtgacagttatgtgtattgtttggaaagaaaatctcatctactaatcatacatgaccaacttgaaatggcttttatacagaaaaacctaagacaattaaactacgtatggattgggcttaactttacctccttgaaaatgacatggacttgggtggatggttctccaatagattcaaagatattcttcataaagggaccagctaaagaaaacagctgtgctgccattaaggaaagcaaaattttctctgaaacctgcagcagtgttttcaaatggatttgtcagtattag

SEQ ID NO: 10 - DNA, 242 bp, Homo sapiens
  source 1..242: mol_type = genomic DNA
attccctatctttgtctactcagcgaacacagggccccgcagagtccgcaaggaacctagagtatgtttcagtgtctccaacgaacaacactgtgtatgcttcagtcactcattcaaacagggaaacagaaatctggacacctagagaaaatgatactatcacaatttactccacaattaatcattccaaagagagtaaacccactttttccagggcaactgcccttgacaatgtcgtgtaa

SEQ ID NO: 11 - DNA, 167 bp, Homo sapiens
  source 1..167: mol_type = genomic DNA
agtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaag

SEQ ID NO: 12 - DNA, 351 bp, Homo sapiens
  source 1..351: mol_type = genomic DNA
aaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaa

SEQ ID NO: 13 - DNA, 210 bp, Homo sapiens
  source 1..210: mol_type = genomic DNA
atccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctga

SEQ ID NO: 14 - DNA, 144 bp, Homo sapiens
  source 1..144: mol_type = genomic DNA
aagcttttaatgataattcatgacagaagggagtttgctaaatttgaaaaggagaaaatgaatgccaaatgggacacgggtgaaaatcctatttataagagtgccgtaacaactgtggtcaatccgaagtatgagggaaaatga

SEQ ID NO: 15 - DNA, 141 bp, Homo sapiens
  source 1..141: mol_type = genomic DNA
aaggctctgatccacctgagcgacctccgggagtacaggcgctttgagaaggagaagctcaagtcccagtggaacaatgataatccccttttcaagagcgccaccacgacggtcatgaaccccaagtttgctgagagttag

SEQ ID NO: 16 - DNA, 144 bp, Homo sapiens
  source 1..144: mol_type = genomic DNA
aaactcctcatcaccatccacgaccgaaaagaattcgctaaatttgaggaagaacgcgccagagcaaaatgggacacagccaacaacccactgtataaagaggccacgtctaccttcaccaatatcacgtaccggggcacttaa

SEQ ID NO: 17 - DNA, 861 bp, Homo sapiens
  source 1..861: mol_type = genomic DNA
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 18 - DNA, 573 bp, Mus musculus
  source 1..573: mol_type = genomic DNA
tataaagttgacttggttctgttctataggcgcatagcggaaagagacgagacactaacagatggtaaaacatatgatgcctttgtgtcttacctgaaagagtgtcatcctgagaataaagaagagtatacttttgctgtggagacgttacccagggtcctggagaaacagtttgggtataagttatgcatatttgaaagagatgtggtgcctggcggagctgttgtcgaggagatccattcactgatagagaaaagccggaggctaatcatcgttctcagccagagttacctgactaacggagccaggcgtgagctcgagagtggactccacgaagcactggtagagaggaagattaagatcatcttaattgagtttactccagccagcaacatcacctttctccccccgtcgctgaaactcctgaagtcctacagagttctaaaatggagggctgacagtccctccatgaactcaaggttctggaagaatcttgtttacctgatgcccgcaaaagccgtcaagccatggagagaggagtcggaggcgcggtctgttctctcagcaccttga

SEQ ID NO: 19 - DNA, 279 bp, Homo sapiens
  source 1..279: mol_type = genomic DNA
aacagggccgcacggcacctgtgcccgccgctgcccacaccctgtgccagctccgccattgagttccctggagggaaggagacttggcagtggatcaacccagtggacttccaggaagaggcatccctgcaggaggccctggtggtagagatgtcctgggacaaaggcgagaggactgagcctctcgagaagacagagctacctgagggtgcccctgagctggccctggatacagagttgtccttggaggatggagacaggtgcaaggccaagatgtga

SEQ ID NO: 20 - DNA, 660 bp, Homo sapiens
  source 1..660: mol_type = genomic DNA
cattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagcccgaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 21 - DNA, 858 bp, Homo sapiens
  source 1..858: mol_type = genomic DNA
agcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaa

SEQ ID NO: 22 - DNA, 1542 bp, Homo sapiens
  source 1..1542: mol_type = genomic DNA
cccctgagcatgcatcagcagcagcagctccagcaccagcagttccagaaggaactggagaagatccagctcctgcagcagcagcagcagcagctgcccttccacccacctggagacacggctcaggacggcgagctcctggacacgtctggcccgtactcagagagctcgggcaccagcagccccagcacgtcccccagggcctccaaccactcgctctgctccggcagctctgcctccaaggctggcagcagcccctccctggaacaagacgatggagatgaggaaaccagcgtggtgatagttgggaaaatttccttctgtcccaaggatgtcctgggccatggagctgagggcacaattgtgtaccggggcatgtttgacaaccgcgacgtggccgtgaagaggatcctccccgagtgttttagcttcgcagaccgtgaggtccagctgttgcgagaatcggatgagcacccgaacgtgatccgctacttctgcacggagaaggaccggcaattccagtacattgccatcgagctgtgtgcagccaccctgcaagagtatgtggagcagaaggactttgcgcatctcggcctggagcccatcaccttgctgcagcagaccacctcgggcctggcccacctccactccctcaacatcgttcacagagacctaaagccacacaacatcctcatatccatgcccaatgcacacggcaagatcaaggccatgatctccgactttggcctctgcaagaagctggcagtgggcagacacagtttcagccgccgatctggggtgcctggcacagaaggctggatcgctccagagatgctgagcgaagactgtaaggagaaccctacctacacggtggacatcttttctgcaggctgcgtcttttactacgtaatctctgagggcagccacccttttggcaagtccctgcagcggcaggccaacatcctcctgggtgcctgcagccttgactgcttgcacccagagaagcacgaagacgtcattgcacgtgaattgatagagaagatgattgcgatggatcctcagaaacgcccctcagcgaagcatgtgctcaaacacccgttcttctggagcctagagaagcagctccagttcttccaggacgtgagcgacagaatagaaaaggaatccctggatggcccgatcgtgaagcagttagagagaggcgggagagccgtggtgaagatggactggcgggagaacatcactgtccccctccagacagacctgcgtaaattcaggacctataaaggtggttctgtcagagatctcctccgagccatgagaaataagaagcaccactaccgggagctgcctgcagaggtgcgggagacgctggggtccctccccgacgacttcgtgtgctacttcacatctcgcttcccccacctcctcgcacacacctaccgggccatggagctgtgcagccacgagagactcttccagccctactacttccacgagcccccagagccccagcccccagtgactccagacgccctctga

SEQ ID NO: 23 - DNA, 795 bp, synthetic construct
  source 1..795: mol_type = other DNA
  misc_feature 1..795: note = Anti-Mesothelin scFv
atggccttaccagtgaccgccttgctcctgccgctggccttgctgctccacgccgccaggccgggatcccaggtacaactgcagcagtctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtggaggcggttcaggcggcggtggctctagcggtggtggatcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgtacggtgctgggacaaagttggaaatcaaagctagc

SEQ ID NO: 24 - DNA, 864 bp, synthetic construct
  source 1..864: mol_type = other DNA
  misc_feature 1..864: note = Anti-CD19 scFv
atggccttaccagtgaccgccttgctcctgccgctggccttgctgctccacgccgccaggccggacatccagatgacacaaaccacctccagcctgtccgcatcccttggagatcgagtcaccattagctgccgggcgtcccaggacatcagcaaatacctcaattggtatcagcaaaaacccgacggcacggtgaagcttctgatataccacacttcacggctgcactcaggcgtgccaagtcggttttcaggatccggctccggcaccgattattctctgaccattagcaatctcgagcaagaggatatcgccacttatttctgtcagcaggggaacaccctcccttatacgtttggtggtgggaccaagttggagattacacgggcagatgccgcccctactgtgagcatatttcctcctagctctaatgggggtggaggctccggtggtggggggagcggcggcggtggctcaggaggcggcggcagcgaggtcaaactgcaagagtctggtccaggcctggtggccccctcccagtctctcagtgtgacctgtaccgtttcaggcgtgtctttgcctgattacggcgtgtcctggataaggcagccaccaagaaagggactggaatggctgggcgttatctggggatcagagacaacttattacaactcagctcttaaatccagactcacgatcattaaggataactctaaatcccaggtgttcctgaagatgaattctctccagacagacgatactgccatctactattgcgctaagcattattattacgggggctcatacgccatggactactggggacagggcaccagcgtgactgtgagttcccctagggctagc

SEQ ID NO: 25 - DNA, 798 bp, synthetic construct
  source 1..798: mol_type = other DNA
  misc_feature 1..798: note = Anti-CD19 scFv
atggccctgcccgtgaccgctctcctgctgcctctggccctgctcctccatgctgccagacccgacatccagatgacacagacaaccagcagcctgtccgcttccctcggagacagggtgacaatttcctgcagggccagccaggacatcagcaagtacctgaactggtaccagcagaaacccgacggcaccgtcaagctcctgatctaccacaccagcagactgcacagcggagtgccttccaggttcagcggcagcggctccggcaccgattactccctgaccattagcaacttagaacaggaggacattgccacctacttttgtcagcagggcaacaccctcccctacacctttggaggcggaaccaagttagaaatcaccggcggcggcggcagcggaggaggaggcagcggaggcggaggctccgaggtgaaactgcaggagagcggccccggactggtcgcccctagccaatccctctccgtcacctgcaccgtgagcggagtgagcctgcctgactacggagtgagctggatcagacagccccctaggaaaggactggaatggctgggcgtgatttggggcagcgagaccacctattacaacagcgccctgaagtccagactgacaatcatcaaggacaatagcaaaagccaagtgtttctgaagatgaacagcctgcagaccgatgacaccgccatctattattgcgccaagcactactactacggaggaagctacgctatggattattggggccaaggcacaagcgtgaccgtcagcagcgcggccgcc

SEQ ID NO: 26 - DNA, 921 bp, synthetic construct
  source 1..921: mol_type = other DNA
  misc_feature 1..921: note = Anti-CD33 scFv
atggccttaccagtgaccgccttgctcctgccgctggccttgctgctccacgccgccaggccgatggaaaaggatacactgttgttgtgggttctgctcctgtgggtgcccggcagcaccggagatattgtgctgacgcagtctcctgcatcactcgccgtgtctctgggccagcgcgctaccatcagctgcagagcctctgaaagtgttgacaattatggaatttctttcatgaattggttccagcagaagcctggccagcccccgaaactcctcatatatgccgcgtctaatcagggctctggggtccctgctagattttctggcagcggctccggcaccgacttcagtctgaatatacatcccatggaagaagacgataccgccatgtacttttgccaacaatctaaggaggtgccttggacgttcggcggcggtacgaagctggaaattaagggcggcgggggaagcggcggggggggatcaggcgggggtggctccggaggcggtggaagtatgggctggagttggatcttccttttccttctttctggtaccgcgggagtgcactctgaggtgcagctccagcagtccggccccgagctcgtcaagcctggggccagtgtcaagatttcctgtaaggcatctggatatacctttacagattacaatatgcattgggtgaaacagtcacatggaaagtcactcgagtggatcggatacatttacccttacaatggaggaaccggatataatcagaagtttaagagcaaggccacactcacggtggacaattcttcatctacagcctacatggatgttcggtctctgacttccgaggatagtgcggtgtattactgcgccaggggacgccccgctatggattactgggggcagggaacctctgtaacagttagctca

SEQ ID NO: 27 - DNA, 1044 bp, synthetic construct
  source 1..1044: mol_type = other DNA
  misc_feature 1..1044: note = Anti-CD123 scFv
atggccttaccagtgaccgccttgctcctgccgctggccttgctgctccacgccgccaggccggacttcgtgatgactcagtctcctagctccctgaccgtgacagccggcgagaaggtgaccatgtcctgcaaatctagtcagagtctgctgaactcaggcaatcagaagaactatctgacatggtacctgcagaagccagggcagccccctaaactgctgatctattgggccagcaccagggaatccggcgtgcccgacagattcaccggctccgggtctggaacagattttactctgaccatttcaagcgtgcaggccgaggacctggctgtgtactattgtcagaatgattacagctatccctacacatttggcgggggaactaagctggaaatcaaaggtggtggtggttctggtggtggtggttccggcggcggcggctccggtggtggtggatccgaggtgcagctgcagcagagtggacccgaactggtgaaacctggcgcctccgtgaaaatgtcttgcaaggctagtgggtacaccttcacagactactatatgaaatgggtgaagcagtcacacgggaagagcctggagtggatcggagatatcattccctctaacggcgccactttctacaatcagaagtttaaaggcaaggctactctgaccgtggaccggagctcctctaccgcctatatgcacctgaacagtctgacatcagaagatagcgctgtgtactattgtacacggtcccatctgctgagagcctcttggtttgcttattggggccaggggacactggtgactgtgagctccgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtg

SEQ ID NO: 28 - DNA, 639 bp, synthetic construct
  source 1..639: mol_type = other DNA
atggccttaccagtgaccgccttgctcctgccgctggccttgctgctccacgccgccaggccgggcatgcgcaccgaggacctgcccaaggccgtggtgttcctggagccccagtggtaccgcgtgctggagaaggactccgtgaccctgaagtgccagggcgcctactcccccgaggacaactccacccagtggttccacaacgagtccctgatctcctcccaggcctcctcctacttcatcgacgccgccaccgtggacgactccggcgagtaccgctgccagaccaacctgtccaccctgtccgaccccgtgcagctggaggtgcacatcggctggctgctgctgcaggccccccgctgggtgttcaaggaggaggaccccatccacctgcgctgccactcctggaagaacaccgccctgcacaaggtgacctacctgcagaacggcaagggccgcaagtacttccaccacaactccgacttctacatccccaaggccaccctgaaggactccggctcctacttctgccgcggcctgttcggctccaagaacgtgtcctccgagaccgtgaacatcaccatcacccagggcctggccgtgcccaccatctcctccttcttcccccccggctaccag

SEQ ID NO: 29 - DNA, 615 bp, synthetic construct
  source 1..615: mol_type = other DNA
atggccttaccagtgaccgccttgctcctgccgctggccttgctgctccacgccgccaggccgcaggccgccgccccccccaaggccgtgctgaagctggagcccccctggatcaacgtgctgcaggaggactccgtgaccctgacctgccagggcgcccgctcccccgagtccgactccatccagtggttccacaacggcaacctgatccccacccacacccagccctcctaccgcttcaaggccaacaacaacgactccggcgagtacacctgccagaccggccagacctccctgtccgaccccgtgcacctgaccgtgctgtccgagtggctggtgctgcagaccccccacctggagttccaggagggcgagaccatcatgctgcgctgccactcctggaaggacaagcccctggtgaaggtgaccttcttccagaacggcaagtcccagaagttctcccacctggaccccaccttctccatcccccaggccaaccactcccactccggcgactaccactgcaccggcaacatcggctacaccctgttctcctccaagcccgtgaccatcaccgtgcaggtgccctccatgggctcctcctcccccatgggc

SEQ ID NO: 30 - DNA, 894 bp, synthetic construct
  source 1..894: mol_type = other DNA
atggccttaccagtgaccgccttgctcctgccgctggccttgctgctccacgccgccaggccgcaggtggacaccaccaaggccgtgatcaccctgcagcccccctgggtgtccgtgttccaggaggagaccgtgaccctgcactgcgaggtgctgcacctgcccggctcctcctccacccagtggttcctgaacggcaccgccacccagacctccaccccctcctaccgcatcacctccgcctccgtgaacgactccggcgagtaccgctgccagcgcggcctgtccggccgctccgaccccatccagctggagatccaccgcggctggctgctgctgcaggtgtcctcccgcgtgttcaccgagggcgagcccctggccctgcgctgccacgcctggaaggacaagctggtgtacaacgtgctgtactaccgcaacggcaaggccttcaagttcttccactggaactccaacctgaccatcctgaagaccaacatctcccacaacggcacctaccactgctccggcatgggcaagcaccgctacacctccgccggcatctccgtgaccgtgaaggagctgttccccgcccccgtgctgaacgcctccgtgacctcccccctgctggagggcaacctggtgaccctgtcctgcgagaccaagctgctgctgcagcgccccggcctgcagctgtacttctccttctacatgggctccaagaccctgcgcggccgcaacacctcctccgagtaccagatcctgaccgcccgccgcgaggactccggcctgtactggtgcgaggccgccaccgaggacggcaacgtgctgaagcgctcccccgagctggagctgcaggtgctgggcctgcagctgcccacccccgtgtggttccac

SEQ ID NO: 31 - DNA, 375 bp, synthetic construct
  source 1..375: mol_type = other DNA
atggccttaccagtgaccgccttgctcctgccgctggccttgctgctccacgccgccaggccggacggcaacgaggagatgggcggcatcacccagaccccctacaaggtgtccatctccggcaccaccgtgatcctgacctgcccccagtaccccggctccgagatcctgtggcagcacaacgacaagaacatcggcggcgacgaggacgacaagaacatcggctccgacgaggaccacctgtccctgaaggagttctccgagctggagcagtccggctactacgtgtgctacccccgcggctccaagcccgaggacgccaacttctacctgtacctgcgcgcccgcgtgtgcgagaactgcatggagatggac

SEQ ID NO: 32 - DNA, 468 bp, synthetic construct
  source 1..468: mol_type = other DNA
atggccttaccagtgaccgccttgctcctgccgctggccttgctgctccacgccgccaggccgaacaagatcctggtgaagcagtcccccatgctggtggcctacgacaacgccgtgaacctgtcctgcaagtactcctacaacctgttctcccgcgagttccgcgcctccctgcacaagggcctggactccgccgtggaggtgtgcgtggtgtacggcaactactcccagcagctgcaggtgtactccaagaccggcttcaactgcgacggcaagctgggcaacgagtccgtgaccttctacctgcagaacctgtacgtgaaccagaccgacatctacttctgcaagatcgaggtgatgtacccccccccctacctggacaacgagaagtccaacggcaccatcatccacgtgaagggcaagcacctgtgcccctcccccctgttccccggcccctccaagcccttc

SEQ ID NO: 33 - DNA, 135 bp, Homo sapiens
  source 1..135: mol_type = genomic DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 34 - DNA, 252 bp, Homo sapiens
  source 1..252: mol_type = genomic DNA
tccacaagaatcaagatcttccctctctgagcaggaatcctttgtgcattgaagactttagattcctctctgcggtagacgtgcacttataagtatttgatggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatct

SEQ ID NO: 35 - DNA, 72 bp, Homo sapiens
  source 1..72: mol_type = genomic DNA
ccattttttttctgctgcttcatcgctgtagccatgggaatccgtttcattattatggtaacaatatggagt

SEQ ID NO: 36 - DNA, 252 bp, Homo sapiens
  source 1..252: mol_type = genomic DNA
cacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaa

SEQ ID NO: 37 - DNA, 73 bp, Homo sapiens
  source 1..73: mol_type = genomic DNA
gtctctttctgcttggtgatggtactcctttttgcagtggacacaggactatatttctctgtgaagacaaaca

SEQ ID NO: 38 - DNA, 252 bp, Homo sapiens
  source 1..252: mol_type = genomic DNA
tgtagaatctaccgcccttctgacaactctgtctctaagtccgtcagattctatctggtggtatctccagcctctgcctccacacagacctcctggactccccgcgacctggtctcttcacagacccagacccagagctgtgtgcctcccactgcaggagccagacaagcccctgagtctccatctaccatccctgtcccttcacagccacagaactccacgctccgccctggccctgcagcccccattgcc

SEQ ID NO: 39 - DNA, 72 bp, Homo sapiens
  source 1..72: mol_type = genomic DNA
ctggtgcctgtgttctgtggactcctcgtagccaagagcctggtgctgtcagccctgctcgtctggtggggg

SEQ ID NO: 40 - DNA, 252 bp, Homo sapiens
  source 1..252: mol_type = genomic DNA
tccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggg

SEQ ID NO: 41 - DNA, 72 bp, Homo sapiens
  source 1..72: mol_type = genomic DNA
gctggtacagtcctcctccttcgggctggattctatgctgtcagctttctctctgtggccgtgggcagcacc

SEQ ID NO: 42 - DNA, 252 bp, Homo sapiens
  source 1..252: mol_type = genomic DNA
ttccccctgggccctgtgaccacagcccacagagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccagaatctccttcgg

SEQ ID NO: 43 - DNA, 72 bp, Homo sapiens
  source 1..72: mol_type = genomic DNA
atgggcctggcctttctagtcctggtggctctagtgtggttcctggttgaagactggctcagcaggaagagg

SEQ ID NO: 44 - DNA, 252 bp, Homo sapiens
  source 1..252: mol_type = genomic DNA
agggaaggggaggcccatgaacgtaggctccctgcagtgcgcagcatcaacggaacattccaggccgactttcctctgggccctgccacccacggagggacctacagatgcttcggctctttccgtgacgctccctacgagtggtcaaactcgagtgatccactgcttgtttccgtcacaggaaacccttcaaatagttggccttcacccactgaaccaagctccaaaaccggtaaccccagacacctacat

SEQ ID NO: 45 - DNA, 69 bp, Homo sapiens
  source 1..69: mol_type = genomic DNA
gttctgattgggacctcagtggtcaaaatccctttcaccatcctcctcttctttctccttcatcgctgg

SEQ ID NO: 46 - DNA, 210 bp, Homo sapiens
  source 1..210: mol_type = genomic DNA
atgaataaacaaagaggaaccttctcagaagtgagtctggcccaggacccaaagcggcagcaaaggaaacctaaaggcaataaaagctccatttcaggaaccgaacaggaaatattccaagtagaattaaatcttcaaaatccttccctgaatcatcaagggattgataaaatatatgactgccaaggtttactgccacctccagagaag

SEQ ID NO: 47 - DNA, 78 bp, Homo sapiens
  source 1..78: mol_type = genomic DNA
ctcactgccgaggtcctaggaatcatttgcattgtcctgatggccactgtgttaaaaacaatagttcttattcctttc

SEQ ID NO: 48 - DNA, 252 bp, Homo sapiens
  source 1..252: mol_type = genomic DNA
gtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 49 - DNA, 72 bp, Homo sapiens
  source 1..72: mol_type = genomic DNA
atctacatctgggcgcccttggccgggacttgtggggtccttctcctgtcactggttatcaccctttactgc

SEQ ID NO: 50 - DNA, 252 bp, Homo sapiens
  source 1..252: mol_type = genomic DNA
gcctcccactactttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacacc

SEQ ID NO: 51 - DNA, 84 bp, Homo sapiens
  source 1..84: mol_type = genomic DNA
attccgtggctcggccacctcctcgtgggcctcagcggggcttttggcttcatcatcttagtgtacttgctgatcaactgcagg

SEQ ID NO: 52 - protein, 465 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAKRGRKKLLYIFKQPFMRPVQTTQEEDGCSCRFPEEEEGGCEL